SEQUENCE LISTING (87 sequences)

SEQ ID NO: 1 - protein, 9 aa, synthetic construct
GGLLQGGGS

SEQ ID NO: 2 - protein, 14 aa, synthetic construct
GGLLQGGGGSGGGS

SEQ ID NO: 3 - protein, 20 aa, synthetic construct
GGLLQGGGGSGGGGSGGGGS

SEQ ID NO: 4 - protein, 13 aa, synthetic construct
GGTLQSPPGGGGS

SEQ ID NO: 5 - protein, 18 aa, synthetic construct
GGTLQSPPGGGGSGGGGS

SEQ ID NO: 6 - protein, 5 aa, synthetic construct
GGLLQ

SEQ ID NO: 7 - protein, 5 aa, synthetic construct
GGTLQ

SEQ ID NO: 8-9 - skipped (2 entries)

SEQ ID NO: 10 - protein, 5 aa, synthetic construct
YRYRQ

SEQ ID NO: 11 - protein, 9 aa, synthetic construct
GGGGSGGGS

SEQ ID NO: 12 - protein, 15 aa, synthetic construct
EPKSSDKTHTCPPCP

SEQ ID NO: 13 - protein, 4 aa, synthetic construct
GGGS

SEQ ID NO: 14 - protein, 8 aa, synthetic construct
GGGSGGGS

SEQ ID NO: 15 - protein, 12 aa, synthetic construct
GGGSGGGSGGGS

SEQ ID NO: 16 - protein, 16 aa, synthetic construct
GGGSGGGSGGGSGGGS

SEQ ID NO: 17 - protein, 5 aa, synthetic construct
GGGGS

SEQ ID NO: 18 - protein, 10 aa, synthetic construct
GGGGSGGGGS

SEQ ID NO: 19 - protein, 15 aa, synthetic construct
GGGGSGGGGSGGGGS

SEQ ID NO: 20 - protein, 20 aa, synthetic construct
GGGGSGGGGSGGGGSGGGGS

SEQ ID NO: 21 - protein, 6 aa, synthetic construct
  VARIANT 3: note = X can be any naturally-occuring amino acid residue or can be absent
  VARIANT 4: note = X can be any naturally-occuring amino acid residue or can be absent
GGXXQG

SEQ ID NO: 22 - protein, 11 aa, synthetic construct
  VARIANT 3: note = X can be any naturally-occuring amino acid residue or can be absent
  VARIANT 4: note = X can be any naturally-occuring amino acid residue or can be absent
  VARIANT 6: note = X can be any naturally-occuring amino acid residue or can be absent
  VARIANT 7: note = X can be any naturally-occuring amino acid residue or can be absent
  VARIANT 9: note = X can be any Q or can be absent
  VARIANT 10: note = X can be Q or can be absent
GGXXQXXQXXG

SEQ ID NO: 23 - protein, 18 aa, synthetic construct
GGTLQSPPCNSTGGGSGG

SEQ ID NO: 24 - protein, 18 aa, synthetic construct
GGTLQSPPGGNSTGGGGS

SEQ ID NO: 25 - protein, 18 aa, synthetic construct
GGTLQSPPGGGNSTGGGS

SEQ ID NO: 26 - protein, 18 aa, synthetic construct
GGTLQSPPGGGGNSTGGS

SEQ ID NO: 27 - protein, 18 aa, synthetic construct
GGTLQAPPGGGCNSTGGS

SEQ ID NO: 28 - protein, 18 aa, synthetic construct
GGTLQAPPGGGGNSTGGS

SEQ ID NO: 29 - protein, 18 aa, synthetic construct
GGPLQAPPGGGGNSTGGS

SEQ ID NO: 30 - protein, 18 aa, synthetic construct
GGTLQSPPGNSTGGGSGG

SEQ ID NO: 31 - protein, 18 aa, synthetic construct
GGRLQAPPGGGGNSTGGS

SEQ ID NO: 32 - protein, 5 aa, synthetic construct
LLQGA

SEQ ID NO: 33 - protein, 5 aa, synthetic construct
WPAQR

SEQ ID NO: 34 - protein, 5 aa, synthetic construct
YEIQR

SEQ ID NO: 35 - protein, 8 aa, synthetic construct
GGTLQSPP

SEQ ID NO: 36 - protein, 8 aa, synthetic construct
GGLLQGPP

SEQ ID NO: 37 - protein, 7 aa, synthetic construct
GGLLQGA

SEQ ID NO: 38 - skipped

SEQ ID NO: 39 - protein, 20 aa, synthetic construct
GGPLQGGGGSGGGGSGGGGS

SEQ ID NO: 40 - protein, 20 aa, synthetic construct
GGTLQGGGGSGGGGSGGGGS

SEQ ID NO: 41 - protein, 20 aa, synthetic construct
GGILQGGGGSGGGGSGGGGS

SEQ ID NO: 42 - protein, 20 aa, synthetic construct
GGSLQGGGGSGGGGSGGGGS

SEQ ID NO: 43 - protein, 20 aa, synthetic construct
GGRLQGGGGSGGGGSGGGGS

SEQ ID NO: 44 - protein, 20 aa, synthetic construct
GGPLQAGGGSGGGGSGGGGS

SEQ ID NO: 45 - protein, 20 aa, synthetic construct
GGTLQAGGGSGGGGSGGGGS

SEQ ID NO: 46 - protein, 20 aa, synthetic construct
GGILQAGGGSGGGGSGGGGS

SEQ ID NO: 47 - protein, 20 aa, synthetic construct
GGRLQAGGGSGGGGSGGGGS

SEQ ID NO: 48 - protein, 20 aa, synthetic construct
GGLLQAGGGSGGGGSGGGGS

SEQ ID NO: 49 - protein, 20 aa, synthetic construct
GGPLQRGGGSGGGGSGGGGS

SEQ ID NO: 50 - protein, 20 aa, synthetic construct
GGTLQRGGGSGGGGSGGGGS

SEQ ID NO: 51 - protein, 20 aa, synthetic construct
GGILQRGGGSGGGGSGGGGS

SEQ ID NO: 52 - protein, 20 aa, synthetic construct
GGSLQRGGGSGGGGSGGGGS

SEQ ID NO: 53 - protein, 20 aa, synthetic construct
GGRLQRGGGSGGGGSGGGGS

SEQ ID NO: 54 - protein, 20 aa, synthetic construct
GGLLQRGGGSGGGGSGGGGS

SEQ ID NO: 55 - protein, 20 aa, synthetic construct
GGTLQDGGGSGGGGSGGGGS

SEQ ID NO: 56 - protein, 20 aa, synthetic construct
GGSLQDGGGSGGGGSGGGGS

SEQ ID NO: 57 - protein, 20 aa, synthetic construct
GGPLQTGGGSGGGGSGGGGS

SEQ ID NO: 58 - protein, 20 aa, synthetic construct
GGTLQTGGGSGGGGSGGGGS

SEQ ID NO: 59 - protein, 20 aa, synthetic construct
GGSLQTGGGSGGGGSGGGGS

SEQ ID NO: 60 - protein, 20 aa, synthetic construct
GGRLQTGGGSGGGGSGGGGS

SEQ ID NO: 61 - protein, 20 aa, synthetic construct
GGLLQTGGGSGGGGSGGGGS

SEQ ID NO: 62 - skipped

SEQ ID NO: 63 - protein, 4 aa, synthetic construct
PLQG

SEQ ID NO: 64 - protein, 4 aa, synthetic construct
TLQG

SEQ ID NO: 65 - protein, 4 aa, synthetic construct
ILQG

SEQ ID NO: 66 - protein, 4 aa, synthetic construct
SLQG

SEQ ID NO: 67 - protein, 4 aa, synthetic construct
RLQG

SEQ ID NO: 68 - protein, 4 aa, synthetic construct
LLQG

SEQ ID NO: 69 - protein, 4 aa, synthetic construct
PLQA

SEQ ID NO: 70 - protein, 4 aa, synthetic construct
TLQA

SEQ ID NO: 71 - protein, 4 aa, synthetic construct
ILQA

SEQ ID NO: 72 - protein, 4 aa, synthetic construct
SLQA

SEQ ID NO: 73 - protein, 4 aa, synthetic construct
RLQA

SEQ ID NO: 74 - protein, 4 aa, synthetic construct
LLQA

SEQ ID NO: 75 - protein, 4 aa, synthetic construct
PLQR

SEQ ID NO: 76 - protein, 4 aa, synthetic construct
TLQR

SEQ ID NO: 77 - protein, 4 aa, synthetic construct
ILQR

SEQ ID NO: 78 - protein, 4 aa, synthetic construct
SLQR

SEQ ID NO: 79 - protein, 4 aa, synthetic construct
RLQR

SEQ ID NO: 80 - protein, 4 aa, synthetic construct
LLQR

SEQ ID NO: 81 - protein, 4 aa, synthetic construct
TLQD

SEQ ID NO: 82 - protein, 4 aa, synthetic construct
SLQD

SEQ ID NO: 83 - protein, 4 aa, synthetic construct
PLQT

SEQ ID NO: 84 - protein, 4 aa, synthetic construct
TLQT

SEQ ID NO: 85 - protein, 4 aa, synthetic construct
SLQT

SEQ ID NO: 86 - protein, 4 aa, synthetic construct
RLQT

SEQ ID NO: 87 - protein, 4 aa, synthetic construct
LLQT

(19) United States
(12) Patent Application Publication
(10) Pub. No.: US 2026/0027222 A1
(43) Pub. Date: Jan. 29, 2026

(54) PEPTIDE LINKERS FOR CONSTRUCTION AND REDUCING AGGREGATION OF FUSION POLYPEPTIDES COMPRISING SUCH

(71) Applicant: SparX Bioscience Limited, Mount Prospect, IL (US)

(72) Inventors: Jichun MA, Vernon Hills, IL (US); Hua JIN, Naperville, IL (US); Qian CHEN, Chicago, IL (US); Jingdong QIN, Woodridge, IL (US); Guidong ZHU, Gurnee, IL (US)

(21) Appl. No.: 19/278,386

(22) Filed: Jul. 23, 2025

Related U.S. Application Data
(60) Provisional application No. 63/674,505, filed on Jul. 23, 2024.

(51) Int. Cl. A61K 47/68; A61K 51/10; C07K 16/22; C07K 16/28; C07K 16/40; C12N 9/10

(52) U.S. Cl. CPC A61K 47/68037; A61K 47/6815; A61K 47/6879; A61K 47/6889; A61K 51/109; A61K 51/1093; C07K 16/22; C07K 16/2827; C07K 16/40; C12N 9/1044; C12Y 204/0203; A61K 2121/00; C07K 2317/31; C07K 2317/569; C07K 2317/92; C07K 2319/00; C12Y 203/02013

(57) ABSTRACT

A non-naturally occurring peptide linker, comprising a site-specific conjugation motif of GGX₁X₂Q, which allows for site-specific conjugation mediated by a transglutaminase; wherein each of X₁ and X₂ independently represents any naturally-occurring amino acid or one of X₁ and X₂ is absent; and wherein the non-naturally occurring peptide has a length of 9-20 amino acids. Also provided herein are fusion polypeptides such as tandem single-domain multi-specific antibodies containing such peptide linkers.

### CROSS REFERENCE TO RELATED APPLICATION

This application claims the benefit of the filing date of U.S. Provisional Application No. 63/674,505, filed on Jul. 23, 2024, the entire contents of which are incorporated by reference herein.

### SEQUENCE LISTING

The instant application contains a Sequence Listing which has been filed electronically in XML format and is hereby incorporated by reference in its entirety. Said XML copy, created on Jul. 21 2025, is named 063667-505001US_Seq-Listing_ST26.xml and is 77,005 bytes in size.

### BACKGROUND OF THE INVENTION

Single-domain antibodies (sdAbs) have garnered significant attention in recent years due to their small size, high stability, and strong binding affinity. These desired properties make sdAbs attractive candidates for various therapeutic and diagnostic applications. sdAbs are often used in constructing tandem single-domain multi-specific antibodies. However, one challenge associated with such multi-specific antibodies is their tendency to aggregate, which can compromise their efficacy and safety. Traditional approaches to mitigate aggregation involve extensive protein engineering and optimization, which can be time-consuming and costly.

Antibody-drug conjugates (ADCs) are complex molecules containing an antibody conjugated to a biologically active agent, which is often named payload. ADCs are biopharmaceutical drugs designed for targeted therapy. Transglutaminase (TGase)-catalyzed conjugation is a conventional approach for linking a payload to an antibody. Such conventional methods often suffer from low specificity and efficiency.

### SUMMARY OF THE INVENTION

The present disclosure is based, at least in part, on the development of specific peptide linkers for use in connecting functional domains of a fusion polypeptide, for example, connecting two single-domain antibody fragments in a tandem single-domain multi-valent and/or multi-specific antibody. The peptide linkers provided herein contains a conjugation motif recognizable by a transglutaminase. Such a motif allows for site-specific conjugation of a payload to a fusion polypeptide containing the peptide linker at the conjugation motif. Further, including the peptide linker in a fusion polypeptide such as a tandem single-domain multi-valent and/or multi-specific antibody would reduce fusion polypeptide aggregation, thereby enhancing its manufacturing and/or formulation efficiency, as well as biological activities such as therapeutic effects.

Accordingly, the present disclosure provides peptide linkers that provide a conjugate site for site-specific linkage of a payload via a reaction catalyzed by a TGase and reduce protein aggregation, fusion polypeptides such as tandem single-domain multi-valent and/or multi-specific antibodies containing such, nucleic acids encoding the peptide linkers or the fusion polypeptides containing such, and host cells comprising the encoding nucleic acids for producing the peptide linkers or the fusion polypeptide comprising such. Also provided herein are conjugates of fusion polypeptides containing the peptide linkers and payloads in a site-specific manner and methods for producing such conjugates via a TGase-catalyzed reaction.

In some aspects, the present disclosure provides a non-naturally occurring peptide linker, comprising a site-specific conjugation motif of GGX₁X₂Q, which allows for site-specific conjugation mediated by a microbial transglutaminase (TGase). In this motif, each of X₁ and X₂ independently represents any naturally-occurring amino acid. Alternatively one of X₁ and X₂ but not both is absent and other one is as defined herein. In some examples, the motif is GGX₁X₂QG (SEQ ID NO: 21) with X₁ and X₂ as defined herein. In some instances, the non-naturally occurring peptide has a length of 9-20 amino acids.

In some embodiments, the non-naturally occurring peptide linker provided herein comprises the amino acid sequence of (i) GGX₁X₂QX₃X₄X₅, in which X₁ is alanine (A), glycine (G), serine(S), threonine (T), proline (P), glutamic acid (E), methionine (M), leucine (L), isoleucine (I), arginine (R), or tyrosine (Y); X₂ is absent or one of leucine (L), isoleucine (I), valine (V), methionine (M), alanine (A), arginine (R), glycine (G), and phenylalanine (F); X₃ is alanine (A), glycine (G), serine(S), threonine (T), proline (P), glutamic acid (E), methionine (M), leucine (L), isoleucine (I), arginine (R), or tyrosine (Y); X₄ is proline (P), glycine (G), or serine(S); and X₅ is proline (P), glycine (G), or serine(S).

In some instances, the non-naturally occurring peptide linker may further comprise a G/S rich motif, for example, (GGGS) n (SEQ ID NOs. 13-16) or (GGGGS) n (SEQ ID NOs. 17-20), in which n can be 1, 2, 3, or 4.

In some examples, the non-naturally occurring peptide linker may comprise the amino acid sequence of:

(SEQ ID NO: 1)
GGLLQGGGS,
(SEQ ID NO: 2)
GGLLQGGGGSGGGS
(SEQ ID NO: 3)
GGLLQGGGGSGGGGSGGGGS,
(SEQ ID NO: 4)
GGTLQSPPGGGGS,
or
(SEQ ID NO: 5)
GGTLQSPPGGGGSGGGGS.

In some instances, the non-naturally occurring peptide linker disclosed herein may contains at least two sites (i.e., two Q residues) for the site-specific conjugation mediated by the microbial transglutaminase. Such a peptide linker may comprise the motif of GGX₁X₂QX₃X₄QX₅X₆G (SEQ ID NO: 22), in which each of X₁-X₄ independently is a naturally-occurring amino acid residue or one or more of X₁-X₄ but not all are absent; each of X₅ and X₆ is Q or absent.

Any of the peptide linkers disclosed herein may be pegylated, i.e., conjugated with a polyethylene glycol (PEG) chain.

In some embodiments, the non-naturally occurring peptide linker of any one of claims 1-8, wherein the linker further comprises an N-glycosylation site. In some instances, the glycosylation site has the motif of NXₐX_{b}, in which Xₐ is any naturally-occurring amino acid residue except for Pro and X_{b} is S or T.

In other aspects, the present disclosure features a fusion polypeptide, comprising a functional segment (e.g., an antibody moiety) and a peptide linker as disclosed herein. In some embodiments, the fusion polypeptide may comprise at least two functional segments and at least one peptide linker as disclosed herein. The at least one peptide linker is located between the two functional segments. In some embodiments, the at least two functional segments are two antibodies. In some specific examples, one or both of the antibodies are single-domain antibodies. In some instances, the fusion polypeptide provided herein may comprise 3-5 single-domain antibodies and 2-4 of the peptide linkers. Each of the peptide linker is located between two adjacent single-domain antibodies. In other specific examples, one of the antibodies is a single-domain antibody and the other antibody is a Fab fragment. The at least one peptide linker is located between the single-domain antibody and one chain of the Fab fragment.

Any of the fusion polypeptides disclosed herein may further comprise a peptide linker as disclosed herein at its N-or C-terminus. In some instances, the peptide linker is located at the C-terminus. In some instances, the terminal peptide linker (e.g., at the C-terminus) comprises the GGX₁X₂Q motif as those disclosed herein. Alternatively, the terminal peptide linker (e.g., at the C-terminus) may has the motif of LLQGA (SEQ ID NO: 32), WPAQR (SEQ ID NO: 33), or YEIQR (SEQ ID NO: 34).

Further, the present disclosure provides a nucleic acid, comprising a nucleotide sequence encoding any of the peptide linkers disclosed herein or the fusion polypeptide comprising such as also disclosed herein. In some embodiments, the nucleic acid is a vector comprising the encoding nucleotide sequence. In some instances, the encoding nucleotide sequence is in operable linkage to a promoter. Also provided herein is a host cell comprising the nucleic acid as disclosed herein.

In yet other aspects, the present disclosure features a conjugate, comprising any of the fusion polypeptides provided herein and a payload. The payload comprises a therapeutic agent or diagnostic and is conjugated covalently at the site-specific conjugation motif of the peptide linker in the fusion polypeptide. The payload may comprise a primary amine group. In some examples, the payload is conjugated to the peptide linker in the fusion polypeptide via a reaction catalyzed by a transglutaminase.

In some instances, the payload further comprises a linker, which contains the primary amine group. Exemplary linkers may comprise the following formula:

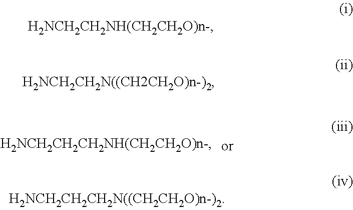

In any of formula (i)-formula (iv) above, n can be an integer of 2-8, inclusive (i.e., 2,3, 4, 5, 6, 7, or 8).

In some embodiments, the payload comprises a therapeutic agent, which can be a radioactive agent (e.g., ²²⁵Ac), a nucleic acid (e.g., an oligonucleotide, such as an RNA molecule), a steroid hormone (e.g., a corticosteroid, such as glucocorticoid), or a chemotherapeutic (e.g., a Tankyrase ½ inhibitor, or a topoisomerase inhibitor, for example, deruxtecan (DXd)). In some instances, the chemotherapeutic may be conjugated to an isotope or a detectable label.

In some embodiments, the conjugate provided herein may comprise at least two therapeutic agents, for example, when linker (ii) or linker (iv) is used in the payload. The two therapeutic agents can be identical. Alternatively, the two therapeutic agents can be different.

In one specific example, the fusion polypeptide comprises a first single-domain antibody specific to PD-L1, a second single-domain antibody specific to VEGF-A, and a peptide linker as provided herein. The peptide linker is located between the first and second single-domain antibodies. In some instances, the payload comprises a topoisomerase inhibitor, for example, deruxtecan (DXd). In some instances, the topoisomerase is conjugated to an isotope.

In another specific example, the fusion polypeptide comprises a first single-domain antibody specific to CAIX, a second single-domain antibody specific to EGFR, and a peptide linker as disclosed herein. The peptide linker is located between the first and second single-domain antibodies. The payload comprises a radionuclide moiety, which optionally can be ²²⁵Ac.

In addition, the present disclosure provides a pharmaceutical composition comprising any of the conjugates disclosed herein and a pharmaceutically acceptable carrier. Also within the present disclosure is a method for treating a disease, comprising administering to a subject in need thereof an effective amount of the pharmaceutical composition, wherein the payload of the conjugate therein comprises a therapeutic agent targeting the disease. Further, provided herein is the pharmaceutical composition for use in treating a target disease, as well as uses of the pharmaceutical composition for manufacturing a medicament for treating the target disease.

In some embodiments, the therapeutic agent is a steroid hormone and the disease is an autoimmune disease. In other embodiments, the therapeutic agent is a Tankyrase ½ inhibitor and the disease is a lung disease, for example, interstitial lung disease (ILD).

In still other aspects, the present disclosure provides a method for preparing a conjugate, the method comprising: contacting a fusion polypeptide as disclosed herein with a payload such as those disclosed herein in the presence of a transglutaminase, which mediates formation of a conjugate comprising the fusion polypeptide and the payload. In some instances, the transglutaminase is immobilized on resins.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1 is a schematic illustration showing a tandem single-domain multi-specific antibody conjugated to a payload.

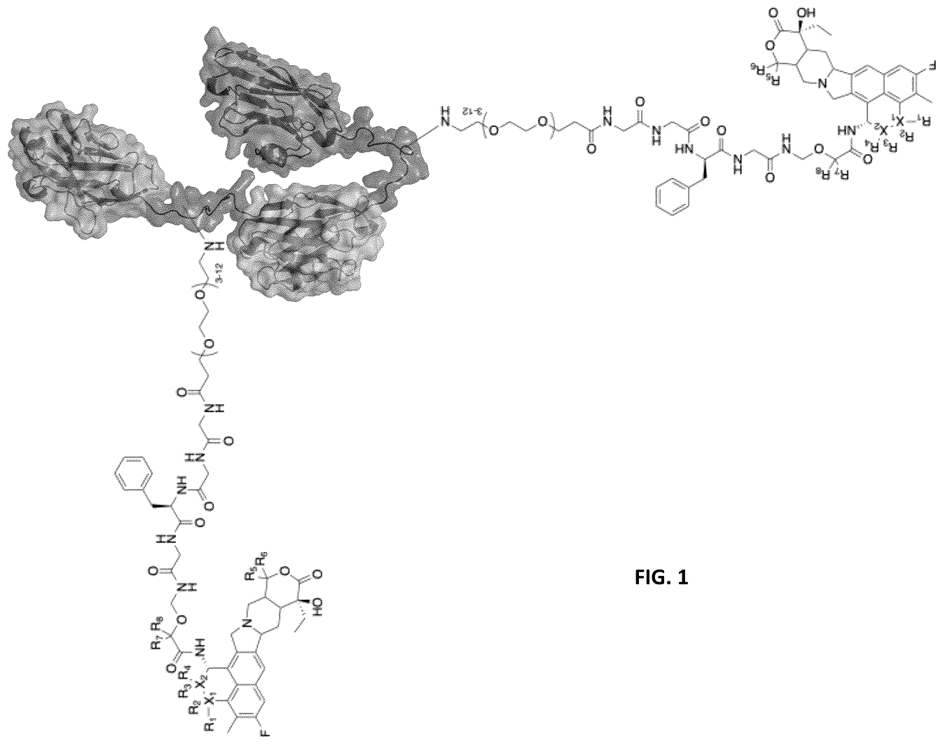

### DETAILED DESCRIPTION OF THE INVENTION

The present disclosure provides specific peptide linkers and fusion polypeptides such as tandem single-domain multivalent and/or multi-specific antibodies comprising such, as well as conjugates comprising the fusion polypeptide and a payload (e.g., a therapeutic agent or a diagnostic agent). The peptide linkers provided herein contain a conjugation motif recognizable by a transglutaminase, which catalyzes the formation of a covalent bond between a primary amine moiety and a gamma-carboxamide moiety. Such a conjugation motif allows for site-specific conjugation of a payload comprising a primary amine moiety to a fusion polypeptide containing the peptide linker at the conjugation motif. Further, including the peptide linker in a fusion polypeptide such as a tandem single-domain multi-valent and/or multi-specific antibody would reduce fusion polypeptide aggregation, thereby enhancing its manufacturing and/or formulation efficiency, as well as biological activities such as therapeutic effects. The present disclosure also provides methods for producing the peptide linkers, the fusion polypeptides, and conjugates comprising such, as well as methods of using such for desired purposes, for example, therapeutic or diagnostic purposes.

### I. Peptide Linkers and Fusion Polypeptides Comprising Such

In some aspects, provided herein are specific peptide linkers (e.g., non-naturally occurring peptide linkers) and fusion polypeptides comprising such. The peptide linkers provide herein provide a motif allowing for site-specific conjugation of a payload comprising a primary amine moiety via a TGase-catalyzed reaction. The peptide linkers provided here may also reduce aggregation of polypeptides when the linkers are used to connecting multiple functional fragments, for example, antibody fragments in constructing fusion polypeptides such as multivalent and/or multi-specific antibodies.

### A. Peptide Linker

The peptide linker provided herein comprises a site-specific conjugation motif that allows for a site-specific conjugation mediated by a transglutaminase. Such a conjugation motif may comprise a site including a glutamine (Q) residue, which is recognizable by a TGase (e.g., a microbial TGase). Mediated by the reaction catalyzed by the TGase, any payload comprising a primary amine moiety can be specifically conjugated to the Q residue via formation of a covalent bond between the primary amine group in the payload and the gamma-carboxamide moiety in the Q residue. In some embodiments, the conjugation motif is located at proper positions in the peptide linker such that the peptide linker can reduce aggregation of a fusion polypeptide (e.g., a tandem single-domain multivalent and/or multi-specific antibody as disclosed herein) comprising such.

Any of the peptide linkers provided herein may have 9-20 amino acid residues in length, for example, any of the integers between 9-20, inclusive, including 9-amino acids and 20-amino acids.

In some embodiments, the peptide linker comprises a site-specific conjugation motif of GGX₁X₂Q (e.g., GGX₁X₂QG; SEQ ID NO: 21). In some examples, each of X₁ and X₂ can be any naturally-occurring amino acid residues. In other examples, one of X₁ and X₂ can be any naturally-occurring amino acid residue and the other one is absent. A naturally-occurring amino acid may be one of the 20 amino acid building blocks of proteins as occurred in nature.

In some instances, the naturally-occurring amino acid may be in a D-amino acid. Alternatively, it may be an L-amino acid.

In some examples, the site-specific conjugation motif may comprise the formula of GGX₁X₂QX₃X₄X₅. In this formula, X₁ can be alanine (A), glycine (G), serine(S), threonine (T), proline (P), glutamic acid (E), methionine (M), leucine (L), isoleucine (I), arginine (R), tyrosine (Y). In some instances, X₂ can be absent. Alternatively, X₂ can be leucine (L), isoleucine (I), valine (V), methionine (M), alanine (A), arginine (R), glycine (G), phenylalanine (F). X₃ can be alanine (A), glycine (G), serine(S), threonine (T), proline (P), glutamic acid (E), methionine (M), leucine (L), isoleucine (I), arginine (R), or tyrosine (Y). Each of X₄ and X₅ independently can be proline (P), glycine (G), or serine(S). In some embodiments, the residues at X₁-X₅ positions of the motifs disclosed herein can be selected to optimize solubility and/or stability of the peptide linker comprising such.

In some embodiments, the peptide linker provided herein may comprise (a) a site-specific conjugation motif of GGLLQ (SEQ ID NO: 6) or GGTLQ (SEQ ID NO: 7), and (b) a motif of (GGGS)n (SEQ ID NO: 13-15) or (GGGGS)n (SEQ ID NO: 17-19), in which n can be 1, 2, or 3. In some examples, the peptide linker may further comprise a segment (e.g., SPP) located between the motifs (a) and (b). Examples of the peptide linkers include, but are not limited to, GGLLQGGGS (SEQ ID NO: 1), GGLLQGGGGSGGGS (SEQ ID NO: 2), GGLLQGGGGSGGGGSGGGGS (SEQ ID NO: 3), GGTLQSPPGGGGS (SEQ ID NO: 4), or GGTLQSPPGGGGSGGGGS (SEQ ID NO: 5).

In other examples, the peptide linker comprises a site-specific conjugation motif of YRYRQ (SEQ ID NO: 10). Such a peptide linker may further comprise a G/S rich fragment as those disclosed herein.

In some instances, the non-naturally occurring peptide linker disclosed herein may contain at least two sites (e.g., 2, 3, or 4 Q residues) for the site-specific conjugation mediated by the microbial transglutaminase. For example, the peptide linker may comprise the motif of GGX₁X₂QX₃X₄QX₅X₆G (SEQ ID NO: 22), in which each of X₁-X₄ independently is a naturally-occurring amino acid residue or one or more of X₁-X₄ but not all are absent; each of X₅ and X₆ is Q or absent. In some instances, one of X₁-X₄ is absent. In other instances, two of

X₁-X₄ is absent. In yet other instances, three of X₁-X₄ is absent. Alternatively or in addition, one of X₅ and X₆ is Q and the other one is absent. In other instances, both X₅ and X₆ are Q residues.

Any of the peptide linkers disclosed herein may be attached to a polyethylene glycol (PEG) chain, i.e., PEGlyated. Pegylation may increase solubility, enhance in vivo half-life, reduce immunogenicity, and/or improve stability of a polypeptide. In some instances, the PEG chain conjugated to the peptide linker may have 3-24 ethylene glycol units.

Alternatively or in addition, any of the peptide linkers disclosed herein may also contain an N-glycosylation site, e.g., NXₐX_{b}, in which Xₐ is any naturally-occurring amino acid residue except for Pro and X_{b} is Ser or Thr. Examples of peptide linkers containing an N-glycosylation site are provided in Tables 1A and 1B below, each of which is within the scope of the present disclosure.

### B. Fusion Polypeptides

Also provided herein are fusion polypeptides comprising at least one peptide linker disclosed herein, which connects two adjacent functional domains in the fusion polypeptide. As used herein, a fusion polypeptide refers to an engineered polypeptide (not naturally-occurring) comprising two or more functional domains from different protein sources. In some instances, the fusion polypeptide provided herein may comprise more than two functional domains, for example, 3, 4, or 5 functional domains, and more than 1 peptide linker (e.g., 2, 3, or 4), each located between two adjacent functional domains. For example, the fusion polypeptide may comprise, from N-terminus to C-terminus, a first functional domain, a first peptide linker, and a second functional domain. In other examples, the fusion polypeptide may comprise, from N-terminus to C-terminus, a first functional domain, a first peptide linker, a second functional domain, a second peptide linker, and a third functional domain. In yet other examples, the fusion polypeptide may comprise, from N-terminus to C-terminus, a first functional domain, a first peptide linker, a second functional domain, a second peptide linker, a third functional domain, a third peptide linker, and a fourth functional domain. Any of the fusion polypeptides provided herein may comprise additional functional domains and additional peptide linkers connecting such. Alternatively or in addition, the fusion polypeptide may further comprise an N-terminal signal peptide. In some examples, all of the peptide linkers included in the fusion polypeptide are those disclosed herein, i.e., containing the site-specific conjugation motif that allows for the site-specific conjugation mediated by a TGase. Alternatively, the fusion polypeptide may also include a conventional G/S-rich flexible linker, such as those provided in Example 1 below.

The fusion polypeptide provided herein may comprise identical functional domains. Alternatively, the fusion polypeptide may comprise different functional domains. Alternatively or in addition, the fusion polypeptide may comprise identical peptide linkers. Alternatively, the fusion polypeptide may comprise different peptide linkers.

### Multivalent and or Multi-Specific Antibodies

In some embodiments, the fusion polypeptide provided herein can be a multivalent and/or multi-specific antibody comprising two or more antibody fragments connected by the peptide linkers also disclosed herein.

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact (e.g., full-length), but also antigen-binding fragments thereof (such as Fab, Fab′, F(ab′)2, Fv), single-chain antibody (scFv), fusion proteins comprising an antibody portion, humanized antibodies, chimeric antibodies, diabodies, single domain antibody (e.g., nanobody), single domain antibodies (e.g., a V_{H} only antibody), multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. A typical antibody molecule comprises a heavy chain variable region (V_{H}) and a light chain variable region (V_{L}), which are usually involved in antigen binding. The V_{H} and V_{L} regions can be further subdivided into regions of hypervariability, also known as “complementarity determining regions” (“CDR”), interspersed with regions that are more conserved, which are known as “framework regions” (“FR”). Each V_{H} and V_{L} is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877;Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs).

In some embodiments, the antibodies described herein specifically bind to the corresponding target antigen or an epitope thereof. An antibody that “specifically binds” to an antigen or an epitope is a term well understood in the art. A molecule is said to exhibit “specific binding” if it reacts more frequently, more rapidly, with greater duration and/or with greater affinity with a particular target antigen than it does with alternative targets. An antibody “specifically binds” to a target antigen or epitope if it binds with greater affinity, avidity, more readily, and/or with greater duration than it binds to other substances. For example, an antibody that specifically (or preferentially) binds to an antigen or an antigenic epitope therein is an antibody that binds this target antigen with greater affinity, avidity, more readily, and/or with greater duration than it binds to other antigens or other epitopes in the same antigen. It is also understood with this definition that, for example, an antibody that specifically binds to a first target antigen may or may not specifically or preferentially bind to a second target antigen. As such, “specific binding” or “preferential binding” does not necessarily require (although it can include) exclusive binding. In some examples, an antibody that “specifically binds” to a target antigen or an epitope thereof may not bind to other antigens or other epitopes in the same antigen (i.e., only baseline binding activity can be detected in a conventional method).

In some embodiments, an antibody for use in making the multivalent and/or multi-specific antibody as described herein has a suitable binding affinity for the target antigen or antigenic epitopes thereof. As used herein, “binding affinity” refers to the apparent association constant or K_{A}. The K_{A} is the reciprocal of the dissociation constant (K_{D}). The antibody described herein may have a binding affinity (K_{D}) of at least 100 nM, 10 nM, 1 nM, 0.1 nM, or lower for the target antigen. An increased binding affinity corresponds to a decreased K_{D}. Higher affinity binding of an antibody for a first antigen relative to a second antigen can be indicated by a higher K_{A} (or a smaller numerical value K_{D}) for binding the first antigen than the K_{A} (or numerical value K_{D}) for binding the second antigen. In such cases, the antibody has specificity for the first antigen (e.g., a first protein in a first conformation or mimic thereof) relative to the second antigen (e.g., the same first protein in a second conformation or mimic thereof; or a second protein). Differences in binding affinity (e.g., for specificity or other comparisons) can be at least 1.5, 2, 3, 4, 5, 10, 15, 20, 37.5, 50, 70, 80, 90, 100, 500, 1000, 10,000 or 105 fold. In some embodiments, any of the anti-CLDN 18.2 antibodies may be further affinity matured to increase the binding affinity of the antibody to the target antigen or antigenic epitope thereof.

Binding affinity (or binding specificity) can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH7.4, 150 mM NaCl, 0.005% (v/v) Surfactant P20). These techniques can be used to measure the concentration of bound binding protein as a function of target protein concentration. The concentration of bound binding protein ([Bound]) is generally related to the concentration of free target protein ([Free]) by the following equation:

$$[ Bound ] = [ Free ] / ( Kd + [ Free ] )$$

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

In some examples, the fusion polypeptide provided herein is a tandem single-domain multivalent and/or multi-specific antibody, which comprises at least two single-domain antibody fragments and at least one peptide linker as disclosed herein connecting the single-domain antibody fragments. As used herein, “a single-domain antibody (sbAb)”, also known as a nanobody, is an antibody fragment having a single antibody variable chain (for example, a heavy variable chain-only antibody), which can bind to a specific antigen. An sbAb typically has a molecule weight of about 12-15 kDa, much smaller than conventional antibodies, which typically have a molecule weight of about 150-160 kDa.

The tandem single-domain multivalent and/or multi-specific antibody provided herein may comprise, from N-terminus to C-terminus, at least the following elements: a first sbAb, a first peptide linker, and a second sbAb. The tandem single-domain multivalent and/or multi-specific antibody may comprise additional sbAb fragments and additional peptide linkers connecting such. In some instances, the tandem single-domain multivalent and/or multi-specific antibody may comprise two sbAb fragments and one peptide linker connecting such. In other instances, the tandem single-domain multivalent and/or multi-specific antibody may comprise three sbAb fragments and two peptide linkers, each connecting two adjacent sbAb segments. In yet other instances, the tandem single-domain multivalent and/or multi-specific antibody may comprise four sbAb fragments and three peptide linkers, each connecting two adjacent sbAb fragments. In still other instances, the tandem single-domain multivalent and/or multi-specific antibody may comprise five sbAb fragments and four peptide linkers, each connecting two adjacent sbAb fragments.

In some instances, the fusion polypeptide provided herein is a multi-chain protein comprising at least one sbAb connected with one chain of a Fab fragment via any of the peptide linkers disclosed herein and the second chain of the Fab fragment. In some examples, the sbAb is connected to the heavy chain of the Fab fragment via the peptide linker. In other examples, the sbAb is connected to the light chain of the Fab fragment.

Any of the tandem single-domain multivalent and/or multi-specific antibody provided herein may comprise at least two identical sbAb fragments. In some instances, all sbAb fragments in the tandem single-domain multivalent and/or multi-specific antibody can be identical. Alternatively, the tandem single-domain multivalent and/or multi-specific antibody provided herein may comprise at least two different sbAb fragments. In some instances, the at least two different sbAb fragments may bind to two different antigens (e.g., multi-specific such as bi-specific). In other instances, the at least two different sbAb fragments may bind to the same antigen but different epitopes thereof.

As used herein, a multivalent antibody refers to an antibody having at least two antigen-binding sites. A multivalent antibody can be specific to one specific antigen. Alternatively, a multivalent antibody can be specific to multiple antigens. As used herein, a multi-specific antibody refers to an antibody having at least two antigen binding fragments, which are specific to different antigens or different epitopes of an antigen. Alternatively or in addition, any of the tandem single-domain multivalent and/or multi-specific antibody provided herein may comprise two or more identical peptide linkers connecting the multiple antibody fragments therein. In some instances, all peptide linkers in a tandem single-domain multivalent and/or multi-specific antibody are identical. Alternatively, the tandem single-domain multivalent and/or multi-specific antibody provided herein may comprise two or more different peptide linkers connecting the multiple antibody fragments therein. In some instances, all peptide linkers in a tandem single-domain multivalent and/or multi-specific antibody are different. In some specific examples, the fusion polypeptide provided herein is a tandem single-domain multi-specific antibody capable of binding to PD-L1 and VEGF-A. Such a multiple-specific antibody may comprise a first single-domain antibody fragment that binds PD-L1, a second single-domain antibody fragment that binds VEGF-A, and a peptide linker as disclosed herein that connects the first and second single-domain antibody fragments. In some instances, the anti-PD-L1/VEGF-A bi-specific antibody may further comprise additional antibody fragments such as additional single-domain antibody fragments, as well as additional peptide linkers connecting two adjacent antibody fragments in the fusion polypeptide. The additional antibody fragments may be specific to PD-L1 and/or VEGF-A. Alternatively, they may be specific to additional antigens.

In some specific examples, the fusion polypeptide provided herein is a tandem single-domain multi-specific antibody capable of binding to CAIX and EGFR. Such a multiple-specific antibody may comprise a first single-domain antibody fragment that binds CAIX, a second single-domain antibody fragment that binds EGFR, and a peptide linker as disclosed herein that connects the first and second single-domain antibody fragments. In some instances, the anti-CAIX/EGFR bi-specific antibody may further comprise additional antibody fragments such as additional single-domain antibody fragments, as well as additional peptide linkers connecting two adjacent antibody fragments in the fusion polypeptide. The additional antibody fragments may be specific to CAIX and/or EGFR. Alternatively, they may be specific to additional antigens.

In some instances, the fusion polypeptide disclosed herein may comprise at least one antibody moiety (e.g., an sbAb) that is specific to human serum albumin protein (HSA). In some instances, the fusion polypeptide disclosed herein may comprise at least one antibody moiety (e.g., an sbAb) that is specific to mouse serum albumin protein (HSA).

In some instances, any of the tandem single-domain multivalent and/or multi-specific antibodies provided herein may include at least one peptide linker as disclosed herein, i.e., containing the site-specific conjugation motif that allows for the site-specific conjugation mediated by a TGase (e.g., a microbial TGase) and at least one conventional G/S-rich flexible linker. Examples are provided in Example 1 below, each of which is within the scope of the present disclosure.

Alternatively or in addition, the tandem single-domain multivalent and/or multi-specific antibodies provided herein may further comprise an Fc domain. In some examples, the Fc domain may be located at the C-terminus of the fusion polypeptide. Alternatively, the Fc fragment may be located between two antibody fragments. The Fc fragment may be connected to its N-terminal antibody fragment via a hinge peptide linker, such as that provided in Table 2B below.

Any of the fusion polypeptides disclosed herein may further comprise a peptide linker as disclosed herein at its N- or C-terminus. In some instances, the peptide linker is located at the C-terminus. Any of the fusion polypeptides disclosed herein may further comprise a peptide linker as disclosed herein at its N-or C-terminus. In some instances, the peptide linker is located at the C-terminus. In some instances, the terminal peptide linker (e.g., at the C-terminus) comprises the GGX₁X₂Q motif as those disclosed herein. Alternatively, the terminal peptide linker (e.g., at the C-terminus) may has the motif of LLQGA (SEQ ID NO: 32), WPAQR (SEQ ID NO: 33), or YEIQR (SEQ ID NO: 34). Exemplary C-terminus peptide linkers are provided in Table 3B below, each of which is within the scope of the present disclosure.

### C. Preparation of Fusion Polypeptides

Any of the fusion polypeptides such as tandem single-domain multivalent and/or multi-specific antibodies can be produced by conventional methods, for example, via recombinant technology. Some examples are provided below.

Generally, a nucleic acid sequence encoding a fusion protein such as a tandem single-domain multivalent and/or multi-specific antibodies comprising any of the peptide linkers provided herein can be cloned into a suitable expression vector in operable linkage with a suitable promoter using methods known in the art. For example, the nucleotide sequence and vector can be contacted, under suitable conditions, with a restriction enzyme to create complementary ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the antibodies.

A variety of promoters can be used for expression of the antibodies described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter.

Regulatable promoters can also be used. Such regulatable promoters include those using the lac repressor from E. coli as a transcription modulator to regulate transcription from lac operator-bearing mammalian cell promoters [Brown, M. et al., (Cell, 49:603-612 (1987)], those using the tetracycline repressor (tetR) [Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)]. Other systems include FK506 dimer, VP16 or p65 using astradiol, RU486, diphenol murislerone, or rapamycin. Inducible systems are available from Invitrogen, Clontech and Ariad.

Regulatable promoters that include a repressor with the operon can be used. In one embodiment, the lac repressor from E. coli can function as a transcriptional modulator to regulate transcription from lac operator-bearing mammalian cell promoters [M. Brown et al., Cell, 49:603-612 (1987); Gossen and Bujard (1992); M. Gossen et al., Natl. Acad. Sci. USA, 89:5547-5551 (1992)] combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy, 10(16): 1392-1399 (2003)). One particular advantage of this tetracycline inducible switch is that it does not require the use of a tetracycline repressor-mammalian cells transactivator or repressor fusion protein, which in some instances can be toxic to cells (Gossen et al., Natl. Acad. Sci. USA, 89:5547-5551 (1992); Shockett et al., Proc. Natl. Acad. Sci. USA, 92:6522-6526 (1995)), to achieve its regulatable effects.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA.

Suitable vectors and methods for producing vectors containing transgenes are well known and available in the art.

Examples of polyadenylation signals useful to practice the methods described herein include, but are not limited to, human collagen I polyadenylation signal, human collagen II polyadenylation signal, and SV40 polyadenylation signal.

One or more vectors (e.g., expression vectors) comprising nucleic acids encoding any of the fusion polypeptides such as the tandem single-domain multi-specific antibodies may be introduced into suitable host cells for producing the fusion polypeptides. The host cells can be cultured under suitable conditions for expression of the fusion polypeptide. Such fusion polypeptides can be recovered by the cultured cells (e.g., from the cells or the culture supernatant) via a conventional method, e.g., affinity purification.

In some embodiments, methods for preparing a fusion polypeptide such as a tandem single-domain multi-specific antibody described herein involve a recombinant expression vector that encodes the fusion polypeptide as also described herein. The recombinant expression vector can be introduced into a suitable host cell (e.g., a dhfr-CHO cell) by a conventional method, e.g., calcium phosphate-mediated transfection. Positive transformant host cells can be selected and cultured under suitable conditions allowing for the expression of the fusion polypeptide, which can be recovered from the cells or from the culture medium.

Standard molecular biology techniques are used to prepare the recombinant expression vector, transfect the host cells, select for transformants, culture the host cells and recovery of the fusion polypeptides such as tandem single-domain multi-specific antibodies from the culture medium.

Any of the nucleic acids encoding the fusion polypeptide such as the tandem single-domain multivalent and/or multi-specific antibodies as described herein, vectors (e.g., expression vectors) containing such; and host cells comprising the vectors are within the scope of the present disclosure.

### II. Conjugates

The present disclosure also provides conjugates comprising any of the fusion polypeptides such as tandem single-domain multivalent and/or multi-specific antibodies as provided herein and a payload, which is covalently conjugated to the fusion polypeptide at the conjugate motif (site-specific) in a peptide linker in the fusion polypeptide. Such site-specific conjugation can be mediated by a TGase, which catalyzes formation of a covalent bond between the fusion polypeptide and a payload.

A. Payloads The payload to be linked to any of the fusion polypeptides such as tandem single-domain multivalent and/or multi-specific antibodies as provided herein can comprise a biologically active moiety (e.g., a therapeutic agent or a diagnostic agent) and a primary amine group, which is the functional moiety for forming a covalent bond catalyzed by the TGase. In some instances, the payload may further comprise a linker moiety, which may comprise the primary amine.

The biologically active moiety can be a suitable molecule, for example, a small molecule therapeutic agent, a radioactive agent, a nucleic acid, which can be double-stranded or single-stranded, a peptide or polypeptide, a lipid, or a saccharide.

In some embodiments, the biologically active moiety can be a chemotherapeutic agent. Any chemotherapeutic agent known in the art can be used for making the conjugates provided herein. In some examples, the chemotherapeutic agent can be a Tankyrase ½ inhibitor. Tankyrase inhibitors (TNKSi) are small molecules that induce Axis inhibition protein ½ stabilization, thereby abrogating Wnt/β-catenin signaling. Exemplary TNKSis include, but are not limited to, XAV939, IWR-1, JW74, and G007-LK. In other examples, the chemotherapeutic agent can be a topoisomerase inhibitor, which can block the activity of topoisomerase. Examples include campothetin (CPT) and derivatives thereof, indenoisoquinoline compounds (e.g., NSC314622, LMP-400, and LMP-776), phenanthridine compounds, and indolocarbazole compounds (e.g., BE-13793C), aminocouimarins, simocyclioners, and quinolones. Specific examples of topoisomerases for use in cancer therapy include, but are not limited to, doxorubicin, daunorubicin, epirubcin, idarubicin, etoposide, teniposide, dexrazoxane, novobiocin, merbarone, and anthrycycline aclarubicin. In some instances, the chemotherapeutic agent may be conjugated to an isotope or a detectable label.

In some embodiments, the biologically active moiety may be a nucleic acid molecule (e.g., an oligonucleotide), for example, a DNA or RNA molecule. The nucleic acid molecule can be double-stranded. Alternatively, the nucleic acid molecule can be single-stranded. In some instances, the nucleic acid molecule may contain one or more modifications, for example, one or more modified bases, one or more modified sugar moiety, and/or one or more modified inter-nucleotide bonds.

In some embodiments, the biologically active moiety may be a steroid hormone. In some examples, the steroid hormone can be a cortisol/corticosterone such as glucocorticoid. Alternatively, the steroid hormone can be an aldosterone such as a mineralocorticoid.

In some embodiments, the biologically active moiety may be a radioactive agent. In some examples, the radioactive agent may be a therapeutic radioactive agent. Examples include ²²⁵Ac, ¹³¹I, ¹⁷⁷Lu, ⁹⁸Sr, ³²P, ¹⁵³Sm, and ²²³Ra. Alternatively, the radioactive agent may be a diagnostic radioactive agent, for example, ⁹⁹Tc.

In some instances, any of the payloads disclosed herein may further comprise a linker in addition to the biologically active moiety. The linker can be attached to the biologically active moiety at one end and has a primary amine group at the other end, which allows for formation of a covalent bond with the gamma-carboxamide moiety of the Q residue in the peptide linker via a TGase-catalyzed reaction. Exemplary linker moieties are as follows:

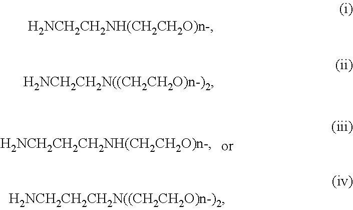

wherein n is an integer of 2-8, inclusive.

In some examples, the linker can be a branched moiety, e.g., linker (ii) and Linker (iv) listed above. Such a branched linker is capable of connecting multiple biologically active moieties. A payload comprising a branched linker may contain multiple copies of a same biologically active moiety. Alternatively, such a payload may contain multiple biologically active moieties, for example, two different therapeutic agents.

### B. Antibody-Drug Conjugates (ADCs)

In some embodiments, provided herein are antibody-drug conjugates (ADCs) comprising one or more antibody moieties and one or more peptide linkers as disclosed herein and a payload comprising a primary amine group. The payload is conjugated to the antibody moiety at the site-specific conjugation motif in the peptide linker via a reaction catalyzed by a TGase enzyme. In some examples, the ADC comprises one antibody moiety fused to a peptide linker as disclosed herein. In other examples, the ADC comprises at least two antibody moieties, which are connected by a peptide linker as disclosed herein.

The antibody moiety in the ADC provided herein can be an antibody of any format, e.g., those disclosed herein. For example, the antibody moiety may be a full-length antibody or a fragment thereof (e.g., Fab). Alternatively, the antibody moiety may be a single chain variable fragment (scFv), which may comprise a V_{H} and V_{L} chains. In some instances, the antibody moiety may be a single-domain antibody fragment.

In specific examples, the antibody moiety in any of the ADCs disclosed herein may be any of the tandem single-domain multivalent and/or multi-specific antibodies disclosed herein. See, e.g., disclosures above. In one example, the ADC comprises the anti-PD-L1/VEGF-A tandem single-domain bi-specific antibody disclosed herein and a payload comprising a topoisomerase inhibitor such as those provided herein. In one example, the topoisomerase inhibitor may be deruxtecan. In some instances, the topoisomerase inhibitor such as deruxtecan may comprise or linked to an isotope (e.g., deuterium). In another example, the ADC comprises the anti-CALIX/EGFR tandem single-domain bi-specific antibody disclosed herein and a payload comprising a radioactive agent such as those provided herein. In one example, the radioactive agent is ²²⁵Ac.

### C. Preparation of Conjugates

The conjugates disclosed herein can be prepared by a reaction catalyzed by a TGase. In nature, transglutaminases are enzymes that catalyze the formation of an isopeptide bond between γ-carboxamide groups (—(C═O)NH2) of glutamine residue side chains and the ε-amino groups (—NH2) of lysine residue side chains. As such, a TGase can catalyze formation of a covalent bond between the gamma-carboxamide moiety in the Q residue of the site-specific conjugation motif in the peptide linker and the primary amine group in the payload, thereby achieving site-specific conjugation of the payload to the fusion polypeptide comprising the peptide linker.

The conjugates provided herein may be produced using a suitable TGase. Exemplary TGase enzymes include human TGase (e.g., keratinocyte TGase, tissue TGase, epidermal TGase, and prostate TGase) and microbial TGase (e.g., mTG, or kalbTG). Such TGase enzymes are well known in the art.

To make the conjugate, any of the fusion polypeptides disclosed herein (e.g., a tandem single-domain multivalent and/or multi-specific antibody as disclosed herein) can be mixed with a suitable payload as also disclosed herein in the presence of a suitable TGase. The mixture thus formed can be incubated under suitable conditions (e.g., temperature) for a suitable period of time to allow for production of the conjugate. In some instances, the TGase may be immobilized to resin, which may be packed into a column. A solution containing the fusion polypeptide may be brought in contact with the TGase-containing resin (e.g., passing through the column) to allow for TGase-mediated reaction and formation of the conjugates. The conjugates thus produced may be isolated/purified by a conventional approach.

### III. Pharmaceutical Compositions

The fusion polypeptides such as tandem single-domain multivalent and/or multispecific antibodies, as well as the encoding nucleic acids or nucleic acid sets, vectors comprising such, or host cells comprising the vectors, or conjugates comprising the fusion polypeptide and a payload as described herein can be mixed with a pharmaceutically acceptable carrier (excipient) to form a pharmaceutical composition for use in treating a target disease. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, are well known in the art. See, e.g., Remington: The Science and Practice of Pharmacy 20th Ed. (2000) Lippincott Williams and Wilkins, Ed. K. E. Hoover.

The pharmaceutical compositions to be used in the present methods can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

In some examples, the pharmaceutical composition described herein comprises liposomes containing the fusion polypeptides (or the encoding nucleic acids) or the conjugates thereof, each of which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545, or by methods disclosed herein. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The fusion polypeptides such as tandem single-domain multivalent and/or multispecific antibodies, as well as the encoding nucleic acids or nucleic acid sets, or conjugates comprising the fusion polypeptide and a payload as described herein may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

In other examples, the pharmaceutical composition described herein can be formulated in sustained-release format. Suitable examples of sustained-release preparations include semipermeable matrices of solid hydrophobic polymers containing the fusion polypeptide or the conjugate thereof, which matrices are in the form of shaped articles, e.g., films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(-)-3-hydroxybutyric acid.

The pharmaceutical compositions to be used for in vivo administration must be sterile. This is readily accomplished by, for example, filtration through sterile filtration membranes. Compositions comprising the fusion polypeptide or the conjugate thereof are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 um, particularly 0.1 and 0.5 pm, and have a pH in the range of 5.5 to 8.0.

The emulsion compositions can be those prepared by mixing a fusion polypeptide or a conjugate thereof with Intralipid™ or the components thereof (soybean oil, egg phospholipids, glycerol and water).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

### IV. Applications of Fusion Polypeptides or Conjugates Thereof

Any of the fusion polypeptides provided herein (e.g., the tandem single-domain multivalent and/or multi-specific antibodies) and any of the conjugates thereof can be used for therapeutic or diagnostic purposes.

In some embodiments, provided herein is a method for treating a disease using a conjugate such as an ADC disclosed herein. To practice the method disclosed herein, an effective amount of the pharmaceutical composition described herein, comprising any of the conjugates disclosed herein can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation or topical routes.

The subject to be treated by the methods described herein can be a mammal, more preferably a human or a non-human primate. Mammals include, but are not limited to, farm animals, sport animals, pets, primates, horses, dogs, cats, mice and rats. A human subject who needs the treatment may be a human patient having, at risk for, or suspected of having a target disease/disorder that can be treated by the payload in the conjugate. For example, a conjugate comprising a payload that includes a steroid hormone such as glucocoticoid can be used for treatment of an autoimmune disease. In another example, a conjugate comprising a payload that includes a Tankyrase ½ inhibitor (e.g., those disclosed herein) can be used to treat lung fibrosis.

In some examples, the conjugate comprises any of the anti-CAIX/EGFR tandem single-domain bispecific antibody and a radioactive agent such as ²²⁵Ac can be used in cancer treatment, for example, treating a hypoxic tumor, which typically express CAIX. Alternatively or in addition such a conjugate may also be used for diagnostic purposes. The conjugate comprises any of the anti-CAIX/EGFR tandem single-domain bispecific antibody and a radioactive agent such as ²²⁵Ac may exhibit one of more of the following advantageous features: (a) improved targeting efficiency via bifunctional single domain; (b) antibody (nanobody) of balanced binding affinity; (c) tissue distribution characterized by analogous nanobody construct; (d) enhanced tumor penetration: penetration to TME in hours vs days; (e) relatively rapid clearance from blood with (t_{1/2}=3-5 h); (f) improved safety and more amendable to radionuclide drug conjugate (RDC) development; and (g) improved tumor-to-background ratio.

A subject having a target disease can be identified by routine medical examination, e.g., laboratory tests, organ functional tests, CT scans, or ultrasounds. In some embodiments, the subject to be treated by the method described herein may be a human patient who has undergone or is subjecting to prior therapy of the disease.

A subject suspected of having any of such target disease/disorder might show one or more symptoms of the disease/disorder. A subject at risk for the disease/disorder can be a subject having one or more of the risk factors for that disease/disorder.

As used herein, “an effective amount” refers to the amount of each active agent required to confer therapeutic effect on the subject, either alone or in combination with one or more other active agents. Determination of whether an amount of the antibody achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a target disease/disorder. Alternatively, sustained continuous release formulations of an antibody may be appropriate. Various formulations and devices for achieving sustained release are known in the art.

In one example, dosages for a fusion polypeptide or a conjugate thereof as described herein may be determined empirically in individuals who have been given one or more administration(s) of the fusion polypeptide or conjugate thereof. Individuals are given incremental dosages of the agonist. To assess efficacy of the agonist, an indicator of the disease/disorder can be followed.

The particular dosage regimen, i.e., dose, timing and repetition, will depend on the particular individual and that individual's medical history, as well as the properties of the individual agents (such as the half-life of the agent, and other considerations well known in the art).

For the purpose of the present disclosure, the appropriate dosage of a fusion polypeptide or a conjugate thereof as described herein will depend on the specific antibody moiety and/or payload moiety employed, the type and severity of the disease/disorder, whether the antibody moiety is administered for preventive or therapeutic purposes, previous therapy, the patient's clinical history and response to the agonist, and the discretion of the attending physician. Typically the clinician will administer an antibody, until a dosage is reached that achieves the desired result. In some embodiments, the desired result is an increase in anti-tumor immune response in the tumor microenvironment. Methods of determining whether a dosage resulted in the desired result would be evident to one of skill in the art. Administration of one or more antibodies can be continuous or intermittent, depending, for example, upon the recipient's physiological condition, whether the purpose of the administration is therapeutic or prophylactic, and other factors known to skilled practitioners. The administration of an antibody may be essentially continuous over a preselected period of time or may be in a series of spaced dose, e.g., either before, during, or after developing a target disease or disorder.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease, or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method.

Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods. In some examples, the pharmaceutical composition is administered intraocularly or intravitreally.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipient is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In one embodiment, any of the fusion polypeptides or conjugates thereof disclosed herein may be administered via site-specific or targeted local delivery techniques. Examples of site-specific or targeted local delivery techniques include various implantable depot sources of the antibody or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568.

In some embodiments, the fusion polypeptides or a conjugate thereof and another suitable therapeutic agent may be administered to a subject in need of the treatment. The fusion polypeptide or the conjugate thereof can also be used in conjunction with other agents that serve to enhance and/or complement the effectiveness of the agents. Treatment efficacy for a target disease/disorder such as those disclosed herein can be assessed by methods well-known in the art. As used herein, the term “combination,” “combined,” and related terms refers to the simultaneous or sequential administration of multiple therapeutic agents in accordance with this disclosure. For example, any of the anti-CLDN 18.2 antibodies or any of the bispecific antibodies as disclosed herein may be administered with another therapeutic agent simultaneously or sequentially in separate unit dosage forms or together in a single unit dosage form.

Any of the fusion polypeptides or conjugates thereof as disclosed here may be used for diagnostic purposes using a conventional method, for example, any immunohistological method known to those of skill in the art (see, e.g., Jalkanen, et al., J. Cell. Biol. 101:976-985 (1985); Jalkanen et al., J. Cell Biol. 105:3087-3096 (1987)).

### V. Kits

The present disclosure also provides kits comprising any of the fusion polypeptides such as tandem single-domain multivalent and/or multi-specific antibodies disclosed herein or any of conjugates comprising such and a payload as also disclosed herein. Such kits can be used for any of the applications of such fusion polypeptides or conjugates as disclosed herein, for example, for use in treating or alleviating a target disease, such as a cancer, a lung disease, or an autoimmune disease as disclosed herein, or for detecting presence or measuring the amount of a target protein or cells expressing the target protein in a biological sample. Such kits can include one or more containers comprising a fusion polypeptide or a conjugate comprising such and a payload as disclosed herein.

In some embodiments, the kit can comprise instructions for use in accordance with any of the methods described herein. The included instructions can comprise a description of administration of the fusion polypeptide or the conjugate thereof to treat, delay the onset, or alleviate a target disease as those described herein. The kit may further comprise a description of selecting an individual suitable for treatment based on identifying whether that individual has the target disease. In still other embodiments, the instructions comprise a description of administering the fusion polypeptide or the conjugate thereof to an individual at risk of the target disease.

The instructions relating to the use of the fusion polypeptide or the conjugate thereof generally include information as to dosage, dosing schedule, and route of administration for the intended treatment. The containers may be unit doses, bulk packages (e.g., multi-dose packages) or sub-unit doses. Instructions supplied in the kits of the present disclosure are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating the disease, such as cancer, immune disorders (e.g., an autoimmune disease), or a lung disease. Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is a fusion polypeptide (e.g., a tandem single-domain multi-specific antibody) or a conjugate thereof as those described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introuction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit, and scope of the present disclosure. All such modifications are intended to be within the scope of the disclosure.

### Example 1: Construction and Production of Tandem Single-Domain Multi-Specific Antibodies

This example illustrates construction and production of multi-specific antibodies comprising multiple single-domain antibodies, at least two of which are connected by a peptide linker that allows for site-specific conjugation as provided herein. In some instances, the tandem single-domain multivalent and/or multi-specific antibodies further comprises an Fc fragment.

A. Structure and Sequences of Tandem Single-Domain Multi-Specific Antibodies Multiple heavy chain-only single-domain antibodies (VHHs, including VHH1, VHH2, VHH3, and VHH4) were used as building blocks to construct exemplary tandem single-domain multi-specific antibodies as provided herein. Adjacent VHH building blocks were linked via either a Gly-Ser flexible linker or the peptide linker that allows for site-specific conjugation as disclosed herein (also named as Q-linkers).

A brief summary of the designs of such tandem single-domain multi-specific antibodies is provided in Tables 1A and 1B below (from N-terminus to C-terminus).

TABLE 1A
Designs of Tandem Single-Domain Multi-Specific Antibodies

| Fusion | | | | | |
|---|---|---|---|---|---|
| Protein | 1^{st} | | 2^{nd} | | 3^{rd} |
| ID | sdAb | Peptide Linker 1 | sdAb | Peptide Linker 2 | sdAb |
| 1 | VHH1 | GGGGSGGGS | VHH1 | GGLLQGGGS | VHH2 |
| | | (SEQ ID NO: 11) | | (SEQ ID NO: 1) | |
| 2 | VHH1 | GGGGSGGGS | VHH1 | GGLLQGGGGSGGGS | VHH2 |
| | | (SEQ ID NO: 11) | | (SEQ ID NO: 2) | |
| 3 | VHH1 | GGGGSGGGS | VHH1 | GGTLQSPPGGGGS | VHH2 |
| | | (SEQ ID NO: 11) | | (SEQ ID NO: 4) | |
| 4 | VHH1 | GGGGSGGGS | VHH1 | GGGGSGGGS | VHH2 |
| | | (SEQ ID NO: 11) | | (SEQ ID NO: 11) | |
| 5 | VHH1 | GGGGSGGGS | VHH1 | GGGGSGGGS | VHH2 |
| | | (SEQ ID NO: 11) | | (SEQ ID NO: 11) | |
| 6 | VHH1 | GGLLQGGGGSGGG | VHH1 | GGLLQGGGS | VHH2 |
| | | GSGGGGS (SEQ ID | | (SEQ ID NO: 1) | |
| | | NO: 3) | | | |
| 7 | VHH1 | GGTLQSPPGGGGSG | VHH1 | GGTLQSPPGGGGS | VHH2 |
| | | GGGS (SEQ ID NO: 5) | | (SEQ ID NO: 4) | |
| 8 | VHH4 | GGLLQGGGGSGGG | VHH4 | GGLLQGGGGSGGGS | VHH2 |
| | | GSGGGGS | | (SEQ ID NO: 2) | |
| | | (SEQ ID NO: 3) | | | |
| 11 | VHH1 | GGTLQSPPCNSTGG | VHH2 | GGTLQSPPGNSTGGGS | VHH1 |
| | | GSGG | | GG | |
| | | (SEQ ID NO: 23) | | (SEQ ID NO: 30) | |
| 12 | VHH1 | GGTLQSPPGGNSTG | VHH2 | GGTLQSPPGGNSTGGG | VHH1 |
| | | GGGS | | GS | |
| | | (SEQ ID NO: 24) | | (SEQ ID NO: 24) | |
| 13 | VHH1 | GGTLQSPPGGGNST | VHH2 | GGTLQSPPGGGNSTGG | VHH1 |
| | | GGGS | | GS | |
| | | (SEQ ID NO: 25) | | (SEQ ID NO: 25) | |
| 14 | VHH1 | GGTLQSPPGGGGNS | VHH2 | GGTLQSPPGGGGNSTG | VHH1 |
| | | TGGS | | GS | |
| | | (SEQ ID NO: 26) | | (SEQ ID NO: 26) | |
| 15 | VHH1 | GGTLQAPPGGGCNS | VHH2 | GGRLQAPPGGGGNST | VHH1 |
| | | TGGS | | GGS | |
| | | (SEQ ID NO: 27) | | (SEQ ID NO: 31) | |
| 16 | VHH1 | GGTLQAPPGGGGNS | VHH2 | GGPLQAPPGGGGNST | VHH1 |
| | | TGGS | | GGS | |
| | | (SEQ ID NO: 28) | | (SEQ ID NO: 29) | |
| 17 | VHH1 | GGPLQAPPGGGGNS | VHH2 | GGPLQAPPGGGGNST | VHH1 |
| | | TGGS | | GGS | |
| | | (SEQ ID NO: 29) | | (SEQ ID NO: 29) | |
| 18 | VHH1 | GGTLQSPPGGGGSG | VHH1 | GGTLQSPPGGGGS | VHH2 |
| | | GGGS (SEQ ID NO: 5) | | (SEQ ID NO: 4) | |
| 19 | VHH1 | GGTLQSPPGGGGSG | VHH1 | GGTLQSPPGGGGS | VHH2 |
| | | GGGS (SEQ ID NO: 5) | | (SEQ ID NO: 4) | |
| 20 | VHH1 | GGTLQSPPGGGGSG | VHH1 | GGTLQSPPGGGGS | VHH2 |
| | | GGGS (SEQ ID NO: 5) | | (SEQ ID NO: 4) | |
| 21 | VHH1 | GGTLQSPPGGGGS | VHH2 | GGTLQSPPGGGGS | VHH1 |
| | | (SEQ ID NO: 4) | | (SEQ ID NO: 4) | |
| 22 | VHH1 | GGTLQSPPGGGGSG | VHH2 | GGTLQSPPGGGGSGG | VHH1 |
| | | GGGS (SEQ ID NO: 5) | | GGS (SEQ ID NO: 5) | |

TABLE 1B
Designs of Tandem Single-Domain Multi-Specific Antibodies

| Fusion | | | | |
|---|---|---|---|---|
| Protein | | 4^{th} | | 5^{th} |
| ID | Peptide Linker 3 | sdAb | Peptide Linker 4 | sdAb |
| 1 | GGLLQGGGS | VHH3 | N/A | N/A |
| | (SEQ ID NO: 1) | | | |
| 2 | GGLLQGGGGSGGGS | VHH3 | N/A | N/A |
| | (SEQ ID NO: 2) | | | |
| 3 | GGTLQSPPGGGGS | VHH3 | N/A | N/A |
| | (SEQ ID NO: 4) | | | |
| 4 | GGGGSGGGS | VHH3 | GGLLQGGGGSGGGGSGG | VHH3 |
| | (SEQ ID NO: 11) | | GGS (SEQ ID NO: 3) | |
| 5 | GGGGSGGGS | VHH3 | GGTLQSPPGGGGSGGGGS | VHH3 |
| | (SEQ ID NO: 11) | | (SEQ ID NO: 5) | |
| 6 | GGLLQGGGS | VHH3 | N/A | N/A |
| | (SEQ ID NO: 1) | | | |
| 7 | GGTLQSPPGGGGS | VHH3 | N/A | N/A |
| | (SEQ ID NO: 4) | | | |
| 8 | GGLLQGGGGSGGGS (SEQ | VHH3 | GGLLQGGGGSGGGGSGG | VHH3 |
| | ID NO: 2) | | GGS (SEQ ID NO: 3) | |
| 11 | N/A | N/A | N/A | N/A |
| 12 | N/A | N/A | N/A | N/A |
| 13 | N/A | N/A | N/A | N/A |
| 14 | N/A | N/A | N/A | N/A |
| 15 | N/A | N/A | N/A | N/A |
| 16 | N/A | N/A | N/A | N/A |
| 17 | GGPLQAPPGGGGNSTGGS | VHH5 | GGPLQAPPGGGGNSTGGS | VHH3 |
| | (SEQ ID NO: 29) | | (SEQ ID NO: 29) | |
| 18 | GGTLQSPPGGGGS (SEQ | VHH3 | GGTLQSPPGGGGSGGGGS | VHH5 |
| | ID NO: 4) | | (SEQ ID NO: 5) | |
| 19 | GGTLQSPPGGGGS (SEQ | VHH6 | GGTLQSPPGGGGSGGGGS | VHH6 |
| | ID NO: 4) | | (SEQ ID NO: 5) | |
| 20 | GGTLQSPPGGGGS (SEQ | VHH7 | GGTLQSPPGGGGSGGGGS | VHH7 |
| | ID NO: 4) | | (SEQ ID NO: 5) | |
| 21 | GGTLQSPPGGGGS | VHH5 | GGTLQSPPGGGGS | VHH3 |
| | (SEQ ID NO: 4) | | (SEQ ID NO: 4) | |
| 22 | GGTLQSPPGGGGSGGGGS | VHH5 | GGTLQSPPGGGGSGGGGS | VHH5 |
| | (SEQ ID NO: 5) | | (SEQ ID NO: 5) | |

### B. Structure and Sequences of Single-Domain Multi-Specific Antibody-Fc Fusion Proteins

Multiple heavy chain-only single-domain antibodies (VHHs, including VHH1 and VHH4) were used as building blocks to construct exemplary tandem single-domain multi-specific antibody-Fc fusion polypeptides as provided herein. Adjacent VHH building blocks were linked via a peptide linker that allows for site-specific conjugation as disclosed herein (also named as Q-linkers). A brief summary of the designs of such tandem single-domain multi-specific antibody-Fc fusion polypeptide is provided in Tables 2A and 2B below (from N-terminus to C-terminus).

TABLE 2A
Designs of Tandem Single-Domain Multi-
Specific Antibody-Fc Fusion Polypeptides

| Fusion | | | | |
|---|---|---|---|---|
| Protein | 1^{st} | | 2^{nd} | |
| ID | sdAb | Peptide Linker 1 | sdAb | Peptide Linker 2 |
| 9 | VHH1 | GGTLQSPPGGGGS | VHH1 | EPKSSDKTHTC |
| | | GGGGS | | PPCP |
| | | (SEQ ID NO: 5) | | (SEQ ID NO: 12) |
| 10 | N/A | N/A | VHH4 | EPKSSDKTHTCPPCP |
| | | | | (SEQ ID NO: 12) |

TABLE 2B
Designs of Tandem Single-Domain Multi-
Specific Antibody-Fc Fusion Polypeptides

| Fusion | | | |
|---|---|---|---|
| Protein | | | 3^{rd} |
| ID | Fc | Peptide Linker 3 | sdAb |
| 9 | IgG1Fc | GGTLQSPPGGGGSGGGGS | VHH3 |
| | | (SEQ ID NO: 5) | |
| 10 | IgG1Fc | GGTLQSPPGGGGSGGGGS | VHH3 |
| | | (SEQ ID NO: 5) | |

C. Structure and Sequences of Single-Domain Multi-Specific Antibodies with C-terminal linkers

The Q-linker can be added to C-terminus of the tandem single-domain multi-specific antibodies and/or between single-domain multi-specific antibodies. A brief summary of the designs of such tandem single-domain multi-specific antibodies is provided in Tables 3A and 3B below (from N-terminus to C-terminus).

TABLE 3A
Designs of Tandem Single-Domain Multi-Specific Antibodies with C-
terminal Q-linker

| Fusion | | | | | |
|---|---|---|---|---|---|
| Protein | 1^{st} | | 2^{nd} | | 3^{rd} |
| ID | sdAb | Peptide Linker 1 | sdAb | Peptide Linker 2 | sdAb |
| 22 | VHH1 | GGGGSGGGS | VHH1 | GGGGSGGGS | VHH2 |
| | | (SEQ ID NO: 11) | | (SEQ ID NO: 11) | |
| 23 | VHH1 | GGGGSGGGS | VHH1 | GGGGSGGGS | VHH2 |
| | | (SEQ ID NO: 11) | | (SEQ ID NO: 11) | |
| 24 | VHH1 | GGGGSGGGS | VHH1 | GGGGSGGGS | VHH2 |
| | | (SEQ ID NO: 11) | | (SEQ ID NO: 11) | |
| 25 | VHH1 | GGGGSGGGS | VHH1 | GGGGSGGGS | VHH2 |
| | | (SEQ ID NO: 11) | | (SEQ ID NO: 11) | |
| 26 | VHH1 | GGGGSGGGS | VHH1 | GGGGSGGGS | VHH2 |
| | | (SEQ ID NO: 11) | | (SEQ ID NO: 11) | |
| 27 | VHH1 | GGGGSGGGS | VHH1 | GGGGSGGGS | VHH2 |
| | | (SEQ ID NO: 11) | | (SEQ ID NO: 11) | |
| 28 | VHH1 | GGGGSGGGS | VHH1 | GGGGSGGGS | VHH2 |
| | | (SEQ ID NO: 11) | | (SEQ ID NO: 11) | |

TABLE 3B
Designs of Tandem Single-Domain Multi-
Specific Antibodies with C-
terminal Q-linker

| Fusion | | | | | |
|---|---|---|---|---|---|
| Protein | Peptide | 4^{th} | Peptide | 5^{th} | C-terminal |
| ID | Linker 3 | sdAb | Linker 4 | sdAb | linker |
| 22 | GGGGSGGGS | VHH3 | N/A | N/A | LLQGA |
| | (SEQ ID NO: 11) | | | | (SEQ ID NO: 32) |
| 23 | GGGGSGGGS | VHH3 | N/A | N/A | WPAQR |
| | (SEQ ID NO: 11) | | | | (SEQ ID NO: 33) |
| 24 | GGGGSGGGS | VHH3 | N/A | N/A | YEIQR |
| | (SEQ ID NO: 11) | | | | (SEQ ID NO: 34) |
| 25 | GGGGSGGGS | VHH3 | N/A | N/A | GGTLQSPP |
| | (SEQ ID NO: 11) | | | | (SEQ ID NO: 35) |
| 26 | GGGGSGGGS | VHH3 | N/A | N/A | GGLLQGPP |
| | (SEQ ID NO: 11) | | | | (SEQ ID NO: 36) |
| 27 | GGGGSGGGS | VHH3 | N/A | N/A | GGLLQGA |
| | (SEQ ID NO: 11) | | | | (SEQ ID NO: 37) |
| 28 | GGGGSGGGS | VHH3 | GGGGSGGGS | VHH3 | GGTLQSPP |
| | (SEQ ID NO: 11) | | (SEQ ID NO: 11) | | (SEQ ID NO: 35) |

### D. Structure and Sequences of Single-Domain Multi-Specific Antibodies With Different Linkers

Bispecific tandem VHH antibodies (VHH1, and VHH3) with single internal linker were used to determine the conjugation efficiency of different linkers.

TABLE 4
Bispecific tandem VHH antibodies

| Fusion | 1^{st} | | 2^{nd} |
|---|---|---|---|
| Protein ID | sdAb | Peptide Linker 1 | sdAb |
| 29 | VHH1 | GGPLQGGGGSGGGGSGGGGS (SEQ ID NO: 39) | VHH3 |
| 30 | VHH1 | GGTLQGGGGSGGGGSGGGGS (SEQ ID NO: 40) | VHH3 |
| 31 | VHH1 | GGILQGGGGSGGGGSGGGGS (SEQ ID NO: 41) | VHH3 |
| 32 | VHH1 | GGSLQGGGGSGGGGSGGGGS (SEQ ID NO: 42) | VHH3 |
| 33 | VHH1 | GGRLQGGGGSGGGGSGGGGS (SEQ ID NO: 43) | VHH3 |
| 34 | VHH1 | GGLLQGGGGSGGGGSGGGGS (SEQ ID NO: 3) | VHH3 |
| 35 | VHH1 | GGPLQAGGGSGGGGSGGGGS (SEQ ID NO: 44) | VHH3 |
| 36 | VHH1 | GGTLQAGGGSGGGGSGGGGS (SEQ ID NO: 45) | VHH3 |
| 37 | VHH1 | GGILQAGGGSGGGGSGGGGS (SEQ ID NO: 46) | VHH3 |
| 38 | VHH1 | GGSLQGGGGSGGGGSGGGGS (SEQ ID NO: 42) | VHH3 |
| 39 | VHH1 | GGRLQAGGGSGGGGSGGGGS (SEQ ID NO: 47) | VHH3 |
| 40 | VHH1 | GGLLQAGGGSGGGGSGGGGS (SEQ ID NO: 48) | VHH3 |
| 41 | VHH1 | GGPLQRGGGSGGGGSGGGGS (SEQ ID NO: 49) | VHH3 |
| 42 | VHH1 | GGTLQRGGGSGGGGSGGGGS (SEQ ID NO: 50) | VHH3 |
| 43 | VHH1 | GGILQRGGGSGGGGSGGGGS (SEQ ID NO: 51) | VHH3 |
| 44 | VHH1 | GGSLQRGGGSGGGGSGGGGS (SEQ ID NO: 52) | VHH3 |
| 45 | VHH1 | GGRLQRGGGSGGGGSGGGGS (SEQ ID NO: 53) | VHH3 |
| 46 | VHH1 | GGLLQRGGGSGGGGSGGGGS (SEQ ID NO: 54) | VHH3 |
| 47 | VHH1 | GGTLQDGGGSGGGGSGGGGS (SEQ ID NO: 55) | VHH3 |
| 48 | VHH1 | GGSLQDGGGSGGGGSGGGGS (SEQ ID NO: 56) | VHH3 |
| 49 | VHH1 | GGPLQTGGGSGGGGSGGGGS (SEQ ID NO: 57) | VHH3 |
| 50 | VHH1 | GGTLQTGGGSGGGGSGGGGS (SEQ ID NO: 58) | VHH3 |
| 51 | VHH1 | GGSLQTGGGSGGGGSGGGGS (SEQ ID NO: 59) | VHH3 |
| 52 | VHH1 | GGRLQTGGGSGGGGSGGGGS (SEQ ID NO: 60) | VHH3 |
| 53 | VHH1 | GGLLQTGGGSGGGGSGGGGS (SEQ ID NO: 61) | VHH3 |
| 54- | VHH1 | GGGGSGGGGSGGGGS (SEQ ID NO: 19) | VHH5 |
| negative | | | |
| control | | | |

### E. Production of Tandem Single-Domain Multi-specific Antibodies and Fc Fusion Proteins

Production of the tandem single-domain multi-specific antibodies, as exemplified by the fusion proteins illustrated in Tables 1A-1B, Tables 2A-2B, and Tables 3A-3B, is provided above.

The exemplary fusion proteins are expressed in either ExpiCHO® or Expi293® cells, following the manufacturer's instructions. Once expressed, the tandem single-domain antibodies (tVHHs) present in the culture media are purified using immobilized A3 resins. Alternatively, cation exchange chromatography (CEX) can be employed, utilizing Praesto Jetted SP35 (Purolite) resins for purification.

### F. Octet BLI Kinetic Binding Assay for Tandem Single-Domain Multi-Specific Antibodies

The kinetic binding of tandem single-domain multi-specific antibodies was measured using an Octet BLI binding assay. The dissociation constant (Kd) for each VHH was measured individually. Streptavidin (SA) biosensors were employed for the assay. Initially, 25 nM biotinylated antigen 1 was loaded onto the tip of the SA sensor, followed by labeling with 25 nM of the tandem format antibody. Serially diluted antigen 3 solutions were then incubated with the antibody-labeled biosensor, and the association rate (ka) and dissociation rate (kdis) of the antibody-antigen 3 binding were recorded. The Kd value for VHH3 (KD-VHH3) was calculated using a 1:1 binding model with global fitting. The Kd values for VHH1 and VHH4 (KD-VHH1/4) were measured using the same method.

Since there is a single VHH2 in the multi-specific single-domain antibodies, the binding kinetics of VHH2 were measured by loading Antigen 2, followed by binding with serially diluted antibody to record the association rate (ka) and dissociation rate (kdis). The Kd value for VHH2 (KD-VHH2) was calculated using a 1:1 binding model with global fitting.

The tandem single-domain multi-specific antibodies with different linkers exhibited similar binding kinetics to their specific antigens. The VHHs in ADC-conjugated multi-specific antibodies also demonstrated similar binding kinetics compared to their parental non-conjugated antibodies. The KD values of the various VHH moieties in the tandem single-domain multi-specific antibodies are shown in Tables 5-7 below.

TABLE 5
KD Values of VHH1 or VHH4

| | KD (M) | ka (1/Ms) | kdis (1/s) |
|---|---|---|---|
| ID1 | 2.66E−10 | 2.80E+05 | 7.45E−05 |
| ID2 | 2.54E−10 | 2.72E+05 | 6.93E−05 |
| ID3 | 1.11E−10 | 3.65E+05 | 4.06E−05 |
| ID4 | <1.0E−12 | 3.52E+05 | <1.0E−07 |
| ID5 | 1.18E−10 | 3.80E+05 | 4.48E−05 |
| ID6 | 9.52E−11 | 3.89E+05 | 3.71E−05 |
| ID7 | 1.20E−10 | 3.55E+05 | 4.26E−05 |
| ID9 | 1.05E−09 | 2.92E+05 | 3.07E−04 |
| ID10 | 4.03E−10 | 5.21E+05 | 2.10E−04 |

TABLE 6
KD Values of VHH3

| | KD (M) | ka (1/Ms) | kdis (1/s) |
|---|---|---|---|
| ID1 | 1.06E−09 | 3.32E+05 | 3.53E−04 |
| ID2 | 1.18E−09 | 3.46E+05 | 4.10E−04 |
| ID3 | 1.35E−09 | 3.28E+05 | 4.42E−04 |
| ID4 | 1.47E−09 | 2.79E+05 | 4.10E−04 |
| ID5 | 1.72E−09 | 2.73E+05 | 4.70E−04 |
| ID6 | 8.90E−10 | 3.50E+05 | 3.11E−04 |
| ID7 | 9.45E−10 | 3.85E+05 | 3.63E−04 |
| ID9 | 1.03E−09 | 4.48E+05 | 4.63E−04 |
| ID10 | 1.05E−09 | 4.11E+05 | 4.31E−04 |

TABLE 7
KD Values of VHH2

| | KD (M) | ka (1/Ms) | kdis (1/s) |
|---|---|---|---|
| ID1 | 1.59E−06 | 1.45E+03 | 2.31E−03 |
| ID2 | 3.40E−07 | 1.01E+04 | 3.44E−03 |
| ID3 | 3.66E−07 | 8.52E+03 | 3.12E−03 |
| ID4 | 2.99E−07 | 7.54E+03 | 2.25E−03 |
| ID5 | 3.28E−07 | 9.38E+03 | 3.07E−03 |
| ID6 | 2.86E−07 | 9.35E+03 | 2.68E−03 |
| ID7 | 2.07E−07 | 1.93E+04 | 3.98E−03 |

### Example 2: Preparation of Fusion Polypeptide-Payload Conjugates

These examples illustrate the methods for preparing conjugated comprising a tandem single-domain multi-specific antibody fusion polypeptide and an exemplary payload.

### A. Conjugation Reaction

The conjugation reaction was carried out either in-solution or on-resin. For an in-solution reaction, the antibody fusion polypeptide in PBS buffer is mixed with the payload stock (50 mM in DMSO) and the purified microbial TGase (MTG) enzyme; for the on-resin reaction, the antibody fusion polypeptide is first bound to A3 protein A or other capturing resin, suspended in PBS, then mix with the payload and MTG. The molecular ratio of the payload to the conjugation site on the antibody fusion polypeptide was maintained between 4:1 and 50:1. The amount of MTG used was 0.3 to 2.4 μg per nmol of the conjugation site. The reaction mixture was incubated at 25-30° C. for 18-40 hours with gentle mixing. Following the reaction, the antibody fusion polypeptide was eluted from the capturing resin (on-resin reaction) or purified using A3 protein A affinity chromatography (in-solution reaction).

Deruxtecan, either native or carrying deuterium, was used as the payload for conjugation to the antibody fusion polypeptide.

In the case of antibodies with fusion protein IDs 1, 2, and 3, 1.0 mg of protein in PBS was first mixed with 10 μL of 50 mM payload, and then 90 μg of MTG was added. The reaction mixture was maintained at 25° C. for 20 hours with gentle shaking. To purify the conjugates, 30 μL of A3 protein A resin was added to each reaction mixture and mixed for 1 hour, followed by thorough washing with PBS and elution with 100 mM glycine pH 3.5. The eluates were subjected to DAR evaluation before being neutralized to a more neutral pH for storage.

For antibodies with fusion protein IDs 4, 5, 6, and 7, 1.0 mg of protein in PBS was first mixed with 10 μL of 50 mM payload, and then 15 μg of MTG was added. The reaction mixture was maintained at 25° C. for 20 hours with gentle shaking. To purify the conjugates, 30 μL of A3 protein A resin was added to each reaction mixture and mixed for 1 hour, followed by thorough washing with PBS and elution with 100 mM glycine pH 3.5. The eluates were subjected to DAR evaluation before being neutralized to a more neutral pH for storage.

For the antibody fusion polypeptides ID 8, 1.0 mg of protein in PBS was first mixed with 20 μL of A3 protein A resin at room temperature for 1 hour. The resin was then washed and resuspended in 200 μL PBS. Following the addition of 5 μL of 50 mM payload and 15 μg of MTG, the reaction mixture was maintained at 25° C. for 22 hours with gentle shaking. To purify the conjugates, the A3 protein A resin was washed with PBS and the conjugate was eluted with 100 mM glycine pH 3.5. The eluates were subjected to DAR evaluation before being neutralized to a more neutral pH for storage.

For the antibody fusion polypeptide ID 9, 0.4 mg of protein in 10 mM Tris pH 6.0 was first mixed with 12 μL of 50 mM payload, and then 40 μg of MTG was added. The reaction mixture was maintained at 25° C. for 40 hours with gentle shaking. To purify the conjugates, 20 μL of A3 protein A resin was added to the reaction mixture and mixed for 1 hour, followed by thorough washing with PBS and elution with 100 mM glycine pH 3.5. The eluates were subjected to DAR evaluation before being neutralized to a more neutral pH for storage.

For the antibody fusion polypeptide ID 10, 1.0 mg of protein in 10 mM Tris buffer, pH 7.2, was first mixed with 75 μL of A3 protein A resin at room temperature for 1 hour. Following the addition of 19 μL of 50 mM payload and 6.8 μg of MTG, the reaction mixture was maintained at 25° C. for 25 hours with gentle shaking. To purify the conjugates, the A3protein A resin was washed with PBS and the conjugate was eluted with 100 mM glycine pH 3.5. The eluates were subjected to DAR evaluation before being neutralized to a more neutral pH for storage.

The reaction setups for the conjugation were summarized in Table 9 below. The exemplary payloads used in this study, for illustration purposes only, are chemotherapeutic topoisomerase I inhibitor (topli) derivatives (see disclosures herein), which are conjugated to the exemplary antibody fusion polypeptides comprising the peptide linker(s) disclosed herein. In some instances, the peptide linkers used herein are pegylated.

### B. Estimation of Drug-to-Antibody Ratio (DAR) by UV-VIS Spectrum

To estimate the drug-to-fusion polypeptide ratio (DAR), a linear relationship between the absorbance at 362 nm (OD₃₆₂) and the concentration of the payload was first established by measuring the OD₃₆₂ values of a series of payload samples with known concentrations. Using this data set, a correlation between OD₃₆₂ and OD₂₈₀ values was established to determine the contribution of payload to the OD₂₈₀.

For the DAR calculation, the OD₃₆₂ and OD₂₈₀ values of a conjugates were measure. The payload concentration was calculated directly from the absorbance at 362 nm. The protein concentration was determined by the extinction coefficient at 280 nm, using the corrected OD₂₈₀ of the conjugates. The corrected OD₂₈₀ was calculated by subtracting the absorbance contribution of the payload at this wavelength from the measured OD₂₈₀ of the conjugates.

Table 10 below shows the theoretical DAR and the calculation of the DAR of various conjugates tested. The results show that in a wide range of payload: reaction sites ratio and wide range of MTG levels, the conjugation occurred efficiently with different peptide linkers. The protein yield from the reaction ranged from 40.0% to 86.8%.

### C. Hydrophobic Interaction Chromatography (HIC) Analysis of Fusion Proteins and ADCs

HIC analysis was performed using the following buffer systems:
Buffer A: 20 mM phosphate (pH 7.0±0.1), 1.5 M ammonium sulfate
Buffer B: 20 mM phosphate (pH 7.0±0.1), 25% isopropyl alcohol

The HIC analysis was carried out on an Agilent HPLC 1260 system equipped with a TOSOH TSKgel Butyl-NPR column (10 cm×4.6 mm). The flow rate was set to 0.4 mL/min using buffers A and B. The column was first equilibrated with 95% buffer A and 5% Buffer B, after injection of the samples, a 2-minute wash phase containing 5% Buffer B was followed, then the antibody was eluted in a gradient phase, increasing Buffer B to 100% over 18 minutes.

The antibody fusion polypeptide-deruxtecan (native or carrying deuterium) conjugates were analyzed in the HIC chromatography assay provided herein. The results show that all the DXd conjugated proteins have a longer retention time than its native protein due to the increase of the hydrophobicity by the incorporation of the payload.

Table 8 shows the retention times for each protein and the relative retention times of conjugated protein compared to the native ones.

TABLE 8
Retention Times of the proteins in HIC analysis

| Fusion | | Retention | | Relative Retention | |
|---|---|---|---|---|---|
| Protein | Theoretical | Time (min) | | Time (min) | |
| ID | DAR | Native | Conjugated | Native | Conjugated |
| 4 | 1 | 15.704 | 16.609 | 1.00 | 1.06 |
| 5 | 1 | 15.695 | 16.515 | 1.00 | 1.05 |
| 6 | 3 | 16.146 | 17.379 | 1.00 | 1.08 |
| 7 | 3 | 15.708 | 17.829 | 1.00 | 1.14 |

TABLE 9
Conjugation Reaction Setup

| Fusion | | Protein | Number of | | | MTG | | |
|---|---|---|---|---|---|---|---|---|
| Protein | | Amount | Conjugation | Payload | Payload:Conjugation | Amount | Temperature | Time |
| ID | Method | (nmol) | Site | (nmol) | Site Ratio | (ug) | (C.°) | (Hour) |
| 1 | In | 19.1 | 2 | 500 | 13.1 | 90 | 25 | 20 |
| | solution | | | | | | | |
| 2 | In | 19.1 | 2 | 500 | 13.1 | 90 | 25 | 20 |
| | solution | | | | | | | |
| 3 | In | 18.9 | 2 | 500 | 13.3 | 90 | 25 | 20 |
| | solution | | | | | | | |
| 4 | In | 15.0 | 1 | 500 | 33.4 | 15 | 25 | 20 |
| | solution | | | | | | | |
| 5 | In | 15.0 | 1 | 500 | 33.4 | 15 | 25 | 20 |
| | solution | | | | | | | |
| 6 | In | 18.8 | 3 | 500 | 8.9 | 15 | 25 | 20 |
| | solution | | | | | | | |
| 7 | In | 18.6 | 3 | 500 | 9.0 | 15 | 25 | 20 |
| | solution | | | | | | | |
| 8 | On-resin | 14.4 | 4 | 250 | 4.4 | 101 | 25 | 22 |
| 9 | In | 3.0 | 4 | 600 | 50.3 | 39 | 25 | 40 |
| | solution | | | | | | | |
| 10 | On-resin | 9.3 | 2 | 950 | 51.1 | 102 | 25 | 25 |
| 11 | In | 17.6 | 2 | 176 | 5 | 42 | 25 | 20 |
| | solution | | | | | | | |
| 12 | In | 10.6 | 2 | 106 | 5 | 25 | 25 | 20 |
| | solution | | | | | | | |
| 13 | In | 10.3 | 2 | 103 | 5 | 25 | 25 | 20 |
| | solution | | | | | | | |
| 14 | In | 18.3 | 2 | 183 | 5 | 44 | 25 | 20 |
| | solution | | | | | | | |
| 15 | In | 32.8 | 2 | 328 | 5 | 79 | 25 | 20 |
| | solution | | | | | | | |
| 16 | In | 31.8 | 2 | 318 | 5 | 76 | 25 | 20 |
| | solution | | | | | | | |
| 17 | In | 14.7 | 4 | 294 | 5 | 35 | 25 | 20 |
| | solution | | | | | | | |
| 22 | In | 19 | 1 | 95 | 5 | 63 | 25 | 20 |
| | solution | | | | | | | |
| 23 | In | 19 | 1 | 95 | 5 | 63 | 25 | 20 |
| | solution | | | | | | | |
| 24 | In | 19 | 1 | 95 | 5 | 63 | 25 | 20 |
| | solution | | | | | | | |
| 25 | In | 19 | 1 | 95 | 5 | 63 | 25 | 20 |
| | solution | | | | | | | |
| 26 | In | 15 | 1 | 75 | 5 | 63 | 25 | 20 |
| | solution | | | | | | | |
| 27 | In | 19 | 1 | 95 | 5 | 63 | 25 | 20 |
| | solution | | | | | | | |
| 28 | In | 19 | 1 | 95 | 5 | 63 | 25 | 20 |
| | solution | | | | | | | |
| 29-53 | On-resin | 36.8 | 1 | 184 | 5 | 11 | 20 | 22 |
| 54 | On-resin | 71.6 | 1 | 358 | 5 | 22 | 20 | 22 |
| 18 | On-resin | 250 | 4 | 1250 | 10 | 161 | 25 | 43 |
| 19 | On-resin | 225 | 4 | 1125 | 10 | 145 | 25 | 43 |
| 20 | In | 33 | 4 | 662 | 5 | 118 | 25 | 24 |
| | solution | | | | | | | |
| 21 | In | 22 | 4 | 440 | 5 | 100 | 25 | 24 |
| | solution | | | | | | | |
| 22 | In | 42 | 4 | 840 | 5 | 151 | 25 | 24 |
| | solution | | | | | | | |

[symbol]

Values of Various Conjugates

| | | | | | Measured | Measured | | |
|---|---|---|---|---|---|---|---|---|
| | | | | | payload | Protein | | Protein |
| | | | | Corrected | concentration | concentration | | Recovery |
| [symbol] | Payload | OD280 | OD362 | OD280 | (nM) | (nM) | DAR | (%) |
| [symbol] | Payload1 | 6.94 | 2.69 | 5.74 | 127.46 | 57.85 | 2.20 | 76.6 |
| [symbol] | Payload1 | 4.01 | 1.33 | 3.43 | 69.34 | 34.52 | 2.01 | 61.0 |
| [symbol] | Payload1 | 7.07 | 2.56 | 5.93 | 121.90 | 59.74 | 2.04 | 74.9 |
| [symbol] | Payload1 | 5.44 | 0.88 | 5.06 | 50.11 | 38.81 | 1.29 | 86.8 |
| [symbol] | Payload1 | 8.52 | 1.34 | 7.93 | 69.76 | 60.86 | 1.15 | 79.4 |
| [symbol] | Payload1 | 7.62 | 3.94 | 5.86 | 180.88 | 58.98 | 3.07 | 66.8 |
| [symbol] | Payload1 | 5.68 | 2.91 | 4.38 | 136.86 | 44.13 | 3.10 | 65.9 |
| [symbol] | Payload1 | 7.57 | 3.62 | 5.95 | 167.20 | 44.65 | 3.74 | 40.0 |
| [symbol] | Payload1 | 5.99 | 1.77 | 5.21 | 88.14 | 22.07 | 3.99 | NA |
| [symbol] | Payload1 | 3.04 | 0.44 | 2.86 | 31.30 | 15.21 | 2.06 | 50.0 |
| [symbol] | Payload2 | 2.79 | 1.07 | 2.47 | 61.49 | 31.25 | 1.97 | 52.0 |
| [symbol] | Payload2 | 1.55 | 0.38 | 1.44 | 21.84 | 18.16 | 1.20 | 60.8 |
| [symbol] | Payload2 | 1.86 | 0.72 | 1.65 | 41.38 | 20.81 | 1.99 | 52.0 |
| [symbol] | Payload2 | 2.47 | 0.97 | 2.18 | 55.75 | 27.58 | 2.02 | 37.0 |
| [symbol] | Payload2 | 4.72 | 1.9 | 4.16 | 109.20 | 52.54 | 2.08 | 51.0 |
| [symbol] | Payload2 | 5.98 | 2.39 | 5.28 | 137.36 | 66.63 | 2.06 | 53.1 |
| [symbol] | Payload3 | 4.91 | 2.37 | 4.21177 | 136.21 | 32.32 | 4.21 | 92.4 |
| [symbol] | Payload4 | 4.97 | 2.50 | 4.23 | 143.68 | 32.49 | 4.42 | 92.9 |
| [symbol] | Payload5 | 4.47 | 2.45 | 3.38 | 117.20 | 25.94 | 4.52 | 34.1 |
| [symbol] | Payload5 | 6.08 | 3.75 | 4.41 | 172.76 | 37.29 | 4.63 | 47.9 |
| [symbol] | [symbol] | [symbol] | [symbol] | [symbol] | [symbol] | [symbol] | [symbol] | [symbol] |

[symbol]

indicates data missing or illegible when filed

[symbol]

R Values of Various Conjugates with C-terminal Q-linker

| | | | | | | Measured | Measured | | |
|---|---|---|---|---|---|---|---|---|---|
| | | | | | | payload | Protein | | |
| | Extinction | | | | Corrected | concentration | concentration | | C-terminal |
| [symbol] | Coefficient | Payload | OD280 | OD362 | OD280 | (nM) | (nM) | DAR | Tag |
| [symbol] | 1.89 | Payload1 | 5.01 | 0.49 | 4.80 | 33.44 | 48.38 | 0.69 | LLQGA |
| | | | | | | | | | (SEQ ID |
| | | | | | | | | | NO: 32) |
| [symbol] | 1.99 | Payload1 | 4.26 | 0.72 | 3.95 | 43.27 | 37.71 | 1.15 | WPAQR |
| | | | | | | | | | (SEQ ID |
| | | | | | | | | | NO: 33) |
| [symbol] | 1.90 | Payload1 | 6.03 | 0.98 | 5.60 | 54.38 | 55.90 | 0.97 | YEIQR |
| | | | | | | | | | (SEQ ID |
| | | | | | | | | | NO: 34) |
| [symbol] | 1.88 | Payload1 | 6.95 | 1.65 | 6.22 | 83.01 | 62.65 | 1.32 | GGTLQSPP |
| | | | | | | | | | (SEQ ID |
| | | | | | | | | | NO: 35) |
| [symbol] | 1.88 | Payload1 | 6.68 | 1.60 | 5.97 | 80.88 | 60.18 | 1.34 | GGLLQGPP |
| | | | | | | | | | (SEQ ID |
| | | | | | | | | | NO: 36) |
| [symbol] | 1.88 | Payload1 | 5.10 | 1.22 | 4.57 | 64.64 | 45.97 | 1.41 | GGLLQGA |
| | | | | | | | | | (SEQ ID |
| | | | | | | | | | NO: 37) |
| [symbol] | [symbol] | [symbol] | [symbol] | [symbol] | [symbol] | [symbol] | [symbol] | [symbol] | GGTLQSPP |
| | | | | | | | | | [symbol] |

[symbol]

indicates data missing or illegible when filed

[symbol]

ed DAR Values of Various Conjugates with Single internal Q-linker

| | | | | | | | Measured | Measured | | |
|---|---|---|---|---|---|---|---|---|---|---|
| | Mol. | | | | | | payload | Protein | | |
| | Wt. | Extinction | | | | Corrected | concentration | concentration | | Internal |
| [symbol] | (kDa) | Coefficient | Payload | OD280 | OD362 | OD280 | (nM) | (nM) | DAR | Q-linker |
| [symbol] | 27.15 | 2.09 | Payload1 | 15.45 | 5.10 | 13.95 | 293.10 | 246.27 | 1.19 | PLQG |
| | | | | | | | | | | (SEQ ID |
| | | | | | | | | | | NO: 63) |
| [symbol] | 27.15 | 2.09 | Payload1 | 6.79 | 2.26 | 6.12 | 129.89 | 108.13 | 1.20 | TLQG |
| | | | | | | | | | | (SEQ ID |
| | | | | | | | | | | NO: 64) |
| [symbol] | 27.15 | 2.09 | Payload1 | 15.44 | 5.10 | 13.94 | 293.10 | 246.09 | 1.19 | ILQG |
| | | | | | | | | | | (SEQ ID |
| | | | | | | | | | | NO: 65) |
| [symbol] | 27.15 | 2.09 | Payload1 | 12.81 | 4.29 | 11.55 | 246.55 | 203.87 | 1.21 | SLQG |
| | | | | | | | | | | (SEQ ID |
| | | | | | | | | | | NO: 66) |
| [symbol] | 27.15 | 2.09 | Payload1 | 17.62 | 6.08 | 15.83 | 349.43 | 279.49 | 1.25 | RLQG |
| | | | | | | | | | | (SEQ ID |
| | | | | | | | | | | NO: 67) |
| [symbol] | 27.15 | 2.09 | Payload1 | 14.52 | 4.82 | 13.10 | 277.01 | 231.31 | 1.20 | LLQG |
| | | | | | | | | | | (positive |
| | | | | | | | | | | control; |
| | | | | | | | | | | SEQ ID |
| | | | | | | | | | | NO: 68) |
| [symbol] | 27.15 | 2.09 | Payload1 | 13.39 | 4.57 | 12.04 | 262.64 | 212.65 | 1.24 | PLQA |
| | | | | | | | | | | (SEQ ID |
| | | | | | | | | | | NO: 69) |

[symbol]

indicates data missing or illegible when filed

| | | | | | | | | | |
|---|---|---|---|---|---|---|---|---|---|
| [symbol] | 2.09 | Payload1 | 9.18 | 2.93 | 8.32 | 168.39 | 146.85 | 1.15 | TLQA |
| | | | | | | | | | (SEQ ID |
| | | | | | | | | | NO: 70) |
| [symbol] | 2.09 | Payload1 | 12.88 | 4.31 | 11.61 | 247.70 | 205.00 | 1.21 | ILQA |
| | | | | | | | | | (SEQ ID |
| | | | | | | | | | NO: 71) |
| [symbol] | 2.09 | Payload1 | 11.44 | 3.83 | 10.31 | 220.11 | 182.07 | 1.21 | SLQA |
| | | | | | | | | | (SEQ ID |
| | | | | | | | | | NO: 72) |
| [symbol] | 2.09 | Payload1 | 15.13 | 5.18 | 13.60 | 297.70 | 240.20 | 1.24 | RLQA |
| | | | | | | | | | (SEQ ID |
| | | | | | | | | | NO: 73) |
| [symbol] | 2.09 | Payload1 | 7.86 | 2.63 | 7.09 | 151.15 | 125.10 | 1.21 | LLQA |
| | | | | | | | | | (positive |
| | | | | | | | | | control; |
| | | | | | | | | | SEQ ID |
| | | | | | | | | | NO: 74) |
| [symbol] | 2.09 | Payload1 | 3.38 | 1.20 | 3.03 | 68.97 | 53.44 | 1.29 | PLQR |
| | | | | | | | | | (SEQ ID |
| | | | | | | | | | NO: 75) |
| [symbol] | 2.09 | Payload1 | 9.95 | 3.32 | 8.97 | 190.80 | 158.42 | 1.20 | TLQR |
| | | | | | | | | | (SEQ ID |
| | | | | | | | | | NO: 76) |
| [symbol] | 2.09 | Payload1 | 6.42 | 2.11 | 5.80 | 121.26 | 102.38 | 1.18 | ILQR |
| | | | | | | | | | (SEQ ID |
| | | | | | | | | | NO: 77) |
| [symbol] | 2.09 | Payload1 | 11.32 | 3.80 | 10.20 | 218.39 | 180.11 | 1.21 | SLQR |
| | | | | | | | | | (SEQ ID |
| | | | | | | | | | NO: 78) |
| [symbol] | [symbol] | [symbol] | [symbol] | [symbol] | [symbol] | [symbol] | [symbol] | [symbol] | [symbol] |

[symbol]

indicates data missing or illegible when filed

| | | | | | | | | | |
|---|---|---|---|---|---|---|---|---|---|
| [symbol] | 2.09 | Payload1 | 10.12 | 3.43 | 9.11 | 197.13 | 160.85 | 1.23 | LLQR |
| | | | | | | | | | (SEQ ID |
| | | | | | | | | | NO: 80) |
| [symbol] | 2.09 | Payload1 | 19.18 | 0.62 | 19.00 | 35.63 | 335.44 | 0.11 | TLQD |
| | | | | | | | | | (SEQ ID |
| | | | | | | | | | NO: 81) |
| [symbol] | 2.09 | Payload1 | 2.56 | 0.81 | 2.32 | 46.55 | 40.99 | 1.14 | SLQD |
| | | | | | | | | | (SEQ ID |
| | | | | | | | | | NO: 82) |
| [symbol] | 2.09 | Payload1 | 13.04 | 3.60 | 11.98 | 206.90 | 211.52 | 0.98 | PLQT |
| | | | | | | | | | (SEQ ID |
| | | | | | | | | | NO: 83) |
| [symbol] | 2.09 | Payload1 | 11.25 | 2.27 | 10.58 | 130.46 | 186.83 | 0.70 | TLQT |
| | | | | | | | | | (SEQ ID |
| | | | | | | | | | NO: 84) |
| [symbol] | 2.09 | Payload1 | 6.39 | 1.19 | 6.04 | 68.39 | 106.64 | 0.64 | SLQT |
| | | | | | | | | | (SEQ ID |
| | | | | | | | | | NO: 85) |
| [symbol] | 2.09 | Payload1 | 10.94 | 3.00 | 10.06 | 172.41 | 177.56 | 0.97 | RLQT |
| | | | | | | | | | (SEQ ID |
| | | | | | | | | | NO: 86) |
| [symbol] | 2.09 | Payload1 | 10.61 | 2.79 | 9.79 | 160.34 | 172.83 | 0.93 | LLQT |
| | | | | | | | | | (SEQ ID |
| | | | | | | | | | NO: 87) |
| [symbol] | 2.23 | Payload1 | 24.92 | 0.11 | 24.89 | 6.32 | 400.62 | 0.02 | GGGS |
| | | | | | | | | | (Negative |
| | | | | | | | | | control; |
| | | | | | | | | | SEQ ID |
| | | | | | | | | | NO: 13) |
| [symbol] | [symbol] | [symbol] | [symbol] | [symbol] | [symbol] | [symbol] | [symbol] | [symbol] | [symbol] |

[symbol]

indicates data missing or illegible when filed

### F. Octet BLI Kinetic Binding Assay for Tandem Single-Domain Multi-Specific Antibody Drug Conjugates

The kinetic binding of tandem single-domain multi-specific antibody drug conjugates (ADCs) was measured using an Octet BLI binding assay. The dissociation constant (Kd) for each VHH was measured individually. Streptavidin (SA) biosensors were employed for the assay.

Initially, 25 nM biotinylated antigen 1 was loaded onto the tip of the SA sensor, followed by labeling with 25 nM of the tandem format antibody. Serially diluted antigen 3 solutions were then incubated with the antibody-labeled biosensor, and the association rate (ka) and dissociation rate (kdis) of the antibody-antigen 3 binding were recorded. The Kd value for VHH3 (KD-VHH3) was calculated using a 1:1 binding model with global fitting. The Kd values for VHH1 and VHH4 (KD-VHH1/4) were measured using the same method.

Since there is a single VHH2 in the multi-specific single-domain antibodies, the binding kinetics of VHH2 were measured by loading Antigen 2, followed by binding with serially diluted antibody to record the association rate (ka) and dissociation rate (kdis). The Kd value for VHH2 (KD-VHH2) was calculated using a 1:1 binding model with global fitting.

The tandem single-domain multi-specific antibodies with different linkers exhibited similar binding kinetics to their specific antigens. The VHHs in ADC-conjugated multi-specific antibodies also demonstrated similar binding kinetics compared to their parental non-conjugated antibodies. The KD values of the various VHH moieties in the tandem single-domain multi-specific antibody drug conjugates are shown in Tables 13-15 below.

TABLE 13
KD Values of VHH1 or VHH4

| | KD (M) | ka (1/Ms) | kdis (1/s) |
|---|---|---|---|
| ID1-Payload1 | 2.44E−10 | 2.44E+05 | 5.95E−05 |
| ID2-Payload1 | 4.00E−10 | 2.06E+05 | 8.23E−05 |
| ID3-Payload1 | 2.90E−10 | 2.21E+05 | 6.41E−05 |
| ID4-Payload1 | 6.32E−11 | 3.67E+05 | 2.32E−05 |
| ID5-Payload1 | 1.89E−10 | 3.55E+05 | 6.71E−05 |
| ID6-Payload1 | 1.64E−10 | 2.61E+05 | 4.29E−05 |
| ID7-Payload1 | 3.97E−10 | 2.43E+05 | 9.65E−05 |
| ID9-Payload1 | 1.35E−09 | 1.80E+05 | 2.44E−04 |
| ID10-Payload1 | 5.52E−10 | 4.23E+05 | 2.34E−04 |

TABLE 14
KD Values of VHH3

| | KD (M) | ka (1/Ms) | kdis (1/s) |
|---|---|---|---|
| ID1-Payload1 | 1.36E−09 | 2.93E+05 | 3.99E−04 |
| ID2-Payload1 | 1.61E−09 | 2.93E+05 | 4.71E−04 |
| ID3-Payload1 | 1.76E−09 | 2.65E+05 | 4.67E−04 |
| ID4-Payload1 | 9.48E−10 | 3.70E+05 | 3.51E−04 |
| ID5-Payload1 | 2.01E−09 | 2.71E+05 | 5.46E−04 |
| ID6-Payload1 | 7.93E−10 | 4.04E+05 | 3.20E−04 |
| ID7-Payload1 | 1.11E−09 | 3.32E+05 | 3.68E−04 |
| ID9-Payload1 | 1.43E−09 | 3.34E+05 | 4.80E−04 |
| ID10-Payload1 | 1.65E−09 | 2.95E+05 | 4.85E−04 |

TABLE 15
KD Values of VHH2

| | KD (M) | ka (1/Ms) | kdis (1/s) |
|---|---|---|---|
| ID1-Payload1 | 1.70E−07 | 9.73E+03 | 1.66E−03 |
| ID2-Payload1 | 1.72E−07 | 9.09E+03 | 1.57E−03 |
| ID3-Payload1 | 1.90E−06 | 1.12E+03 | 2.12E−03 |
| ID4-Payload1 | 1.91E−07 | 8.64E+03 | 1.65E−03 |
| ID5-Payload1 | 1.68E−07 | 9.26E+03 | 1.55E−03 |
| ID6-Payload1 | 1.81E−06 | 9.87E+02 | 1.78E−03 |
| ID7-Payload1 | 9.44E−08 | 9.10E+03 | 8.59E−04 |

### G. ADC Cytotoxicity Assay

To determine cytotoxicity effect of the conjugated ADC, target cells were seeded in 96-well tissue culture plates at a density of 2,000 cells per well in 96 well plate with an appropriate culture medium. The cells were incubated overnight at 37° C. in a humidified atmosphere containing 5% CO₂ to allow adherence and recovery. The following day, cells were treated with varying concentrations of the ADC and incubated for six (6) days under standard culture conditions.

At the end of the treatment period, Cell Counting Kit-8 (CCK-8) reagent was added to each well and absorbance at 450 nm (OD450) were measured according to manufacturer's instructions. Half-maximal inhibitory concentration (IC₅₀) values were calculated using nonlinear regression analysis with GraphPad Prism or equivalent software. Table 16 below shows cytotoxicity of selected ADCs.

TABLE 16
Cytotoxicity of ADCs

| | IC₅₀ (nM) |
|---|---|
| ID16-Payload5 | 0.4974 |
| ID15-Payload4 | 0.3027 |
| ID15-Payload3 | 0.3370 |
| ID20-Payload3 | 0.2441 |
| ID21-Payload3 | 0.3005 |
| ID22-Payload3 | 0.2054 |

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A non-naturally occurring peptide linker, comprising a site-specific conjugation motif of GGX1X2Q , which allows for site-specific conjugation mediated by a microbial transglutaminase;
wherein each of X1 and X2 independently represents any naturally-occurring amino acid or one of X1 and X2 but not both is absent; and
wherein the non-naturally occurring peptide linker has a length of 9-20 amino acids.

2. The non-naturally occurring peptide linker of claim 1, which comprises the amino acid sequence of GGX1X2QX3X4X5;
wherein:
X1 is selected from the group consisting of alanine (A), glycine (G), serine(S), threonine (T), proline (P), glutamic acid (E), methionine (M), leucine (L), isoleucine (I), arginine (R), and tyrosine (Y);
X2 is selected from the group consisting of leucine (L), isoleucine (I), valine (V), methionine (M), alanine (A), arginine (R), glycine (G), and phenylalanine (F);
X3 is selected from the group consisting of alanine (A), glycine (G), serine(S), threonine (T), proline (P), glutamic acid (E), methionine (M), leucine (L), isoleucine (I), arginine (R), and tyrosine (Y);
X4 is selected from the group consisting of proline (P), glycine (G), and serine(S); and
X5 is selected from the group consisting of proline (P), glycine (G), and serine(S).

3. The non-naturally occurring peptide linker of claim 2, wherein X3 is G, and optionally wherein X4 and X5 are absent.

4. The non-naturally occurring peptide linker of claim 1, which further comprises a G/S rich fragment, optionally wherein the G/S rich fragment is (GGGS)n (SEQ ID NOs: 13-16) or (GGGGS)n (SEQ ID NOs. 17-20), in which n is an integer between 1 to 4,inclusive.

5. The non-naturally occurring peptide linker of claim 1, which comprises the amino acid sequence of:
(SEQ ID NO: 1)
GGLLQGGGS,
(SEQ ID NO: 2)
GGLLQGGGGSGGGS,
(SEQ ID NO: 3)
GGLLQGGGGSGGGGSGGGGS,
(SEQ ID NO: 4)
GGTLQSPPGGGGS,
or
(SEQ ID NO: 5)
GGTLQSPPGGGGSGGGGS

6. The non-naturally occurring peptide linker of claim 1, wherein the peptide linker contains at least two sites for the site-specific conjugation mediated by the microbial transglutaminase.

7. The non-naturally occurring peptide linker of claim 6, which comprises the motif of GGX₁X₂QX₃X₄QX5X6G (SEQ ID NO: 22), in which each of X1-X4 independently is a naturally-occurring amino acid residue or one or more of X1-X4 but not all are absent; each of X5 and X6 is Q or absent.

8. The non-naturally occurring peptide linker of claim 1, wherein the peptide linker is pegylated.

9. The non-naturally occurring peptide linker of claim 1, wherein the peptide linker further comprises an N-glycosylation site, which has the motif of NXaXb, in which Xa is any naturally-occurring amino acid residue except for Pro and Xb is S or T.

10. A fusion polypeptide, comprising at least one functional segment and at least one peptide linker set forth in claim 1.

11. The fusion polypeptide of claim 10, wherein the fusion polypeptide comprises at least two functional segments and the at least one peptide linker is located between the two functional segments.

12. The fusion polypeptide of claim 11, wherein the fusion polypeptide comprises at least two functional segments, which are two antibodies.

13. The fusion polypeptide of claim 12, wherein one or both of the antibodies are single-domain antibodies.

14. The fusion polypeptide of claim 13, wherein the fusion polypeptide comprises 3-5 single-domain antibodies, and 2-4 of the peptide linkers; and wherein one of the peptide linker is located between two adjacent single-domain antibodies in the fusion polypeptide.

15. The fusion polypeptide of claim 12, wherein one of the antibodies is a single-domain antibody and the other one of the antibodies is a Fab fragment; and wherein the at least one peptide linker is located between the single-domain antibody and one chain of the Fab fragment.

16. The fusion polypeptide of claim 10, wherein the fusion polypeptide comprises at its C-terminus a peptide linker (a) comprising a site-specific conjugation motif of GGX1X2Q , which allows for site-specific conjugation mediated by a microbial transglutaminase; wherein each of X1 and X2 independently represents any naturally-occurring amino acid or one of X1 and X2 but not both is absent; or (b) set forth as LLQGA (SEQ ID NO: 32), WPAQR (SEQ ID NO: 33), or YEIQR (SEQ ID NO: 34); and
wherein the peptide linker has a length of 9-20 amino acids.

17. A nucleic acid, comprising a nucleotide sequence encoding the peptide linker set forth in claim 1, or a fusion polypeptide comprising such.

18. (canceled)

19. A host cell, comprising the nucleic acid of claim 17.

20. A conjugate, comprising a fusion polypeptide set forth in claim 10 and a payload, wherein the payload comprises a therapeutic agent or a diagnostic agent and a primary amine group; and wherein the payload is conjugated covalently at the site-specific conjugation motif of the peptide linker in the fusion polypeptide.

21-28. (canceled)

29. A method for treating a disease, comprising administering to a subject in need thereof an effective amount of a pharmaceutical composition comprising the conjugate of claim 20, wherein the payload of the conjugate therein comprises a therapeutic agent targeting the disease.

30. (canceled)

31. A method for preparing a conjugate, the method comprising: contacting a fusion polypeptide set forth in claim 5 with a payload comprising a therapeutic agent or a diagnostic agent and a primary amine group in the presence of a transglutaminase, which mediates formation of a conjugate comprising the fusion polypeptide and the payload.

32. (canceled)
